(19) United States
(12) Patent Application Publication
(10) Pub. No.: US 2023/0257770 A1
(43) Pub. Date: Aug. 17, 2023

(54) Process for Making Adenoassociated Viral Vectors

(71) Applicants: Oxford Genetics Limited, Oxford Oxfordshire (GB); Oxford University Innovation Limited, Oxford (GB)

(72) Inventors: Ryan Cawood, Oxford Oxfordshire (GB); Weiheng Su, Oxford Oxforshire (GB)

(21) Appl. No.: 17/796,303

(22) PCT Filed: Feb. 3, 2021
(86) PCT No.: PCT/GB2021/050235

§ 371 Date: Jul. 29, 2022

(30) Foreign Application Priority Data
Feb. 4, 2020 (GB) 2001486.6
Jun. 17, 2020 (GB) 2009241.7
Jul. 14, 2020 (GB) 2010835.5
Jul. 23, 2020 (GB) 2011437.7

(51) Int. Cl. C12N 15/86; C07K 14/075

(52) U.S. Cl. CPC C12N 15/86; C07K 14/075; C12N 2750/14143; C12N 2710/10341

(57) ABSTRACT

The invention relates to a nucleic acid molecule encoding at least one AAV Rep polypeptide, wherein one or more of the AAV (p5), (p19) and (p40) promoters have been modified to reduce or eliminate expression of one or more of the Rep polypeptides, or the nucleic acid molecule does not encode functional (Rep52) or (Rep40) polypeptides, or the nucleic acid molecule does not encode a functional adenovirus inhibitor sequence. The invention also relates to a process for producing recombinant AAV vectors through the use of a 2-adenovirus system, wherein all of the genes required for AAV replication and packaging (i.e. an AAV rep sequence of the invention, AAV cap and the AAV transfer vector comprising a transgene) may be encoded within two adenoviruses.

### CROSS-REFERENCE

This application is a 371 U.S. national phase of PCT/GB2021/050235, filed Feb. 3, 2021, which claims priority from GB application no. 2001486.6, filed Feb. 4, 2020; GB application no. 2009241.7, filed Jun. 17, 2020; GB application no. 2010835.3, filed Jul. 14, 2020; and GB application no. 2011437.7, filed Jul. 23, 2020, all which are incorporated by reference in their entirety.

### STATEMENT REGARDING SEQUENCE LISTING

A computer readable form of the Sequence Listing is filed with this application by electronic submission and is incorporated into this application by reference in its entirety. The sequence listing submitted herewith is contained in the file created Mar. 6, 2023, entitled “22-1002-WO—US_Sequence_Listing_ST25.txt” and 44 kilobytes in size.

### FIELD OF THE INVENTION

The invention relates to a nucleic acid molecule encoding at least one AAV Rep polypeptide, wherein one or more of the AAV p5, p19 and p40 promoters have been modified to reduce or eliminate expression of one or more of the Rep polypeptides, or the nucleic acid molecule does not encode functional Rep52 or Rep40 polypeptides, or the nucleic acid molecule does not encode a functional adenovirus inhibitor sequence.

The invention also relates to a process for producing recombinant AAV vectors through the use of a 2-adenovirus system, wherein all of the genes required for AAV replication and packaging (i.e. an AAV rep sequence of the invention, AAV cap and the AAV transfer vector comprising a transgene) may be encoded within two adenoviruses.

### BACKGROUND OF THE INVENTION

Adeno-associated viruses (AAVs) are single-stranded DNA viruses that belong to the Parvoviridae family. This virus is capable of infecting a broad range of host cells, including both dividing and non-dividing cells. In addition, it is a non-pathogenic virus that generates only a limited immune response in most patients.

Over the last few years, vectors derived from AAVs have emerged as an extremely useful and promising mode of gene delivery. This is owing to the following properties of these vectors:
AAVs are small, non-enveloped viruses and they have only two native genes (rep and cap). Thus they can be easily manipulated to develop vectors for different gene therapies. This is achieved by the removal of the rep and cap genes in the AAV genome and replacing these sequences with exogenous sequences (transgenes) that may provide therapeutic benefit to a patient.
AAV particles are not easily degraded by shear forces, enzymes or solvents. This facilitates easy purification and final formulation of these viral vectors.
AAVs are non-pathogenic and have a low immunogenicity. The use of these vectors further reduces the risk of adverse inflammatory reactions. Unlike other viral vectors, such as lentivirus, herpes virus and adenovirus, AAVs are harmless and are not thought to be responsible for causing any human disease.
Genetic sequences up to approximately 4500 bp can be delivered into a patient using AAV vectors.
Whilst wild-type AAV vectors have been shown to sometimes insert genetic material into human chromosome 19, this property is generally eliminated from most AAV gene therapy vectors by removing rep and cap genes from the viral genome. In such cases, the virus remains in an episomal form within the host cells. These episomes remain intact in non-dividing cells, while in dividing cells they are lost during cell division.

The native AAV genome comprises two genes each encoding multiple open reading frames (ORFs): the rep gene encodes non-structural proteins that are required for the AAV life-cycle and site-specific integration of the viral genome; and the cap gene encodes the structural capsid proteins.

In addition, these two genes are flanked by inverted terminal repeat (ITR) sequences consisting of 145 bases that have the ability to form hairpin structures. These hairpin sequences are required for the primase-independent synthesis of a second DNA strand and the integration of the viral DNA into the host cell genome.

In order to eliminate any integrative capacity of the virus, recombinant AAV vectors remove rep and cap from the DNA of the viral genome. To produce such vectors, the desired transgene(s), together with a promoter(s) to drive transcription of the transgene(s), is inserted between the inverted terminal repeats (ITRs); and the rep and cap genes are provided in trans in a second plasmid. Helper genes such as adenovirus E4, E2a and VA genes are also provided. rep, cap and helper genes may be provided on additional plasmids that are transfected into cells.

Traditionally, the production of AAV vectors has been achieved through a number of different routes.

Initially, AAV was generated using wild-type (WT) Adenovirus serotype 5 whilst transfecting cells with plasmids encoding the rep and cap genes and the AAV genome. This allowed the WT adenovirus to provide a number of factors in trans that facilitated virus replication. However, there are a number of limitations to this approach: for example, each batch of AAV must be separated from the Adenoviral (AV) particles after manufacture to provide a pure product and ensuring that all Ad5 has been removed is challenging. Moreover, the fact that during production the cell is devoting huge resource to the production of Adenoviral particles rather than AAV is also undesirable.

In other systems, stable packing cells lines expressing the rep and cap genes have been used. In such systems, the rep and cap genes are integrated into the cell genomes, hence obviating the need for plasmid-based rep and cap genes. However, these genes are usually only integrated at low frequency (e.g. 1-2 copies per cell) due to their inherent toxicity. These systems require the infection with adenoviral vectors.

More recently, the adenovirus-based systems have been replaced with plasmids encoding the sections of the Adenovirus genome required for AAV production. Whilst this has solved some of the concerns over Adenovirus particles being present in the final virus preparation, a number of issues remain. These include the requirement to pre-manufacture sufficient plasmid for transfection into the production cell line and the inherently inefficient process of transfection itself. The yields from these systems are also lower than those using Ad5-based approaches.

Encoding the AAV genes (rep and cap) and the AAV transfer vector, within adenoviruses have been explored extensively in the past (Fisher, K. J et al., 1996; Liu, X. L. et al., 1999). While the AAV genome and the AAV Cap DNA sequences are tolerated when they are inserted within the genome of an adenovirus, the AAV Rep proteins and DNA sequences are lethal to adenoviruses and inhibit their replication. Despite the toxicity of the AAV Rep on adenovirus replication, a few publications have reported success in producing adenoviruses encoding the AAV Rep polypeptide. However, these adenoviruses are generally unstable, compounded with low titre in production, or loss of the rep gene following multiple passages of the virus (Zhang, H. G et al. 2001; Zhang, X and Li, C-Y, Mol. Ther. 2001).

Inhibition of adenovirus by AAV Rep is caused by two main mechanisms. Firstly, AAV Rep proteins are potent inhibitors of adenovirus promoters (including MLP, E2B, E4) (Timpe J. M. et al., 2006). Secondly, an adenovirus inhibitor sequence is encoded within the AAV rep DNA (located within the p40 promoter that is normally used by the virus for driving expression of the cap genes). Publications have shown that the AAV rep gene can be tolerated within an adenovirus by scrambling this ‘inhibitory’ p40 DNA sequence (Sitaraman, V. et al., 2011; Weger, S. et al., J. Virol. 2016).

### SUMMARY OF THE INVENTION

The current invention is based on a combination of steps which have enabled the AAV rep gene to be stably encoded into an adenoviral vector; the AAV rep gene has never been successfully inserted and maintained in an adenoviral vector before. In particular, in some embodiments, the AAV rep gene promoters p5, p19 and p40 have all been modified or removed. The p5 promoter has been removed to reduce expression of the Rep 78 and Rep 68 polypeptides, and hence reduce their toxicity; the p19 promoter has been removed to stop expression of the Rep52 and Rep40 polypeptides; and the AV inhibitor sequence within the p40 promoter has been removed or modified, or its transcription prevented.

When using AV to manufacture AAV, it is preferable only to need to modify one AV for each genetically-distinct desired AAV. However, the combined molecular size of a rep gene, cap gene and transfer AAV genome would exceed the packaging capacity of an E1/E3-deleted AV vector.

The inventors have now discovered that advantages may be obtained by using two AVs. Given that a genetically-distinct AAV may contain a unique capsid and transfer AAV genome, one aspect of the invention relates to contacting a cell with two AV vectors: one containing the AAV Rep-coding sequence and the other containing a Cap-coding sequence and a transfer AAV genome sequence (the latter defined as a sequence flanked by AAV inverted terminal repeats).

The inventors have also determined that the presence of a Rep-coding sequence and AAV ITRs sequences within the same AV are detrimental to AV growth. Whilst such AVs can be recovered, their yield is typically 5-10 fold lower than when AVs contain each sequence independently.

In another aspect of the invention, therefore, the Rep-coding sequence does not contain a promoter driving its expression; and its transcriptional orientation matches that of the E2A, E2B and E4 transcription units in the AV genome. This ensures that the Rep polypeptide is expressed at a low, base or minimal level in order to reduce toxicity to the cell; and that it is not transcribed to a high level via transcriptional read-through from the strong E1A promoter embedded within the adenovirus packaging signal element.

In yet a further aspect, a first AV containing a rep gene may optionally also encode a protein that can transcriptionally activate a promoter in a second AV that is driving the expression of the cap gene. This allows transcription of the cap gene to only be induced when both AVs are present within the same cell, thereby reducing the burden of expressing the AAV cap gene during AV manufacture.

It is an object of the invention to provide a nucleic acid molecule comprising an AAV rep gene, wherein the rep gene promoters have been modified or eliminated to make the integration of the nucleic acid molecule in an adenoviral vector, and/or subsequent expression thereof, less toxic.

It is another object of the invention to provide an adenoviral vector and a host cell comprising a nucleic acid molecule of the invention.

Other objects of the invention are to provide processes for producing modified host cells and adenoviral vectors comprising nucleic acid molecules of the invention; and processes for producing AAV.

### DESCRIPTION OF THE DRAWINGS

FIG. 1: This shows the structure of the wild-type AAV genome, illustrating the various AAV promoters within the rep gene.

FIGS. 2A and 2B show that AAV Rep proteins can repress basal transcription from the adenovirus major late promoter. The results shown in FIGS. 2A and 2B support that high expression of the Rep proteins inhibit the activity of the adenoviral Major late Promoter or modified forms of the Major Late Promoter that contain a TetR binding site.

FIGS. 3A and 3B show that AAV Rep DNA is stably integrated and replicated with the adenovirus genome. FIG. 3A shows that the frequency of AAV Rep coding DNA and the adenoviral Hexon DNA are present in equal numbers, demonstrating that the Rep DNA insertion into the adenoviral genome is stable. FIG. 3B shows the adenoviral vector encoding the Rep coding sequence and also an AAV genome expressing EGFP.

FIGS. 4A and 4B support that co-infection with TERA vectors significantly increases the production of AAV in HEK293 cells compare to helper-free plasmid transfection method. FIG. 4A shows that the titres achieved are considerably higher than those achieved with the triple transfection or helper-free approach. FIG. 4B shows a schematic representation of the adenoviral genomes containing AAV components for TERA-AAV-Rep and TERA-AAV-Cap.

FIG. 5 shows that co-infection with TERA vectors encoding the AAV transfer genome and AAV2 cap with TERA encoding AAV Rep78-68 significantly increases the production of AAV in HEK293 cells compare to helper-free plasmid transfection method.

FIG. 6 shows that production of AAV by either co-infection of HEK293 cells with TERA-AAV and TERA-RepCap or Helper-free plasmid transfection. Results show total AAV infectious particles and the ratio of transducing particles relative to total genome containing AAV particles. Quantitation is via a modified TCID50 method, where serial dilutions of AAV are added to HEK293 cells and GFP positive cells are counted at the lowest dilutions. The AAV titres is then reverse calculated according to the TCID50 method.

FIG. 7 demonstrates that the invention does not interfere with a repressible adenoviral system as disclosed in WO2019/020992. Total infectious adenoviral particles are shown, demonstrating that when the AAV Rep and Cap genes are integrated into adenoviral vectors this approach can still be used to prevent adenoviral contamination of an AAV preparation. Quantitation is by QPCR against adenoviral hexon sequences.

FIG. 8 demonstrates that the incorporation of the AAV Rep, Cap and genome into adenoviral vectors allows plasmid free AAV production and significantly improved yields compared to plasmid-based production methods. Quantitation is using QPCR against the EGFP expression cassette in the AAV vectors.

FIG. 9 shows serial passage of an adenoviral vector encoding AAV rep and AAV Cap genes. QPCR quantitation for these sequences relative to the adenovirus hexon sequences demonstrates that these genes have been stably inserted and can be propagated over extended periods.

FIG. 10 illustrates a Western blot showing AAV Rep expression from TERA-RepCap showing expression of all main isoforms of AAV Rep.

FIG. 11 illustrates a Western blot of either cell infected with adenoviral vectors encoding AAV Rep (TERA-RepCap, TERA2.0 or TERA2.0+Dox) or AAV2 reference material.

FIG. 12 demonstrates that AAV production is higher when cells are infected with two adenoviral vectors (TERA2.0), one encoding Rep and Cap from AAV serotype 5 and one encoding an AAV genome, in comparison to the helper free method.

FIG. 13 demonstrates that AAV production is higher when cells are infected with two adenoviral vectors (TERA2.0), one encoding Rep and Cap from AAV serotype 6 and one encoding an AAV genome, in comparison to the helper free method.

FIG. 14 demonstration that AAV production is higher when cells are infected with two adenoviral vectors (TERA2.0), one encoding Rep and Cap from AAV serotype 9 and one encoding an AAV genome, in comparison to the helper free method.

FIG. 15A shows that the incorporation of the Rep coding sequence into an adenoviral vector significantly increases AAV yields in comparison to a plasmid encoding Rep or the triple plasmid/helper free approach. FIG. 15B demonstrates that even when the Rep promoter is driven from a strong CMV promoter, the incorporation into an adenoviral vector is significantly superior for AAV productivity.

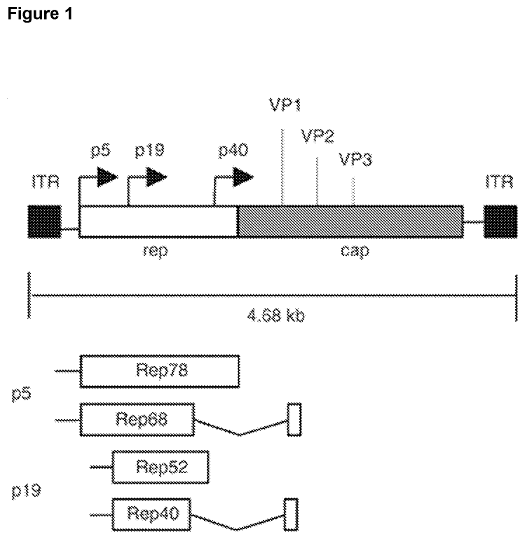

### DETAILED DESCRIPTION OF THE INVENTION

In one embodiment, the invention provides a nucleic acid molecule, wherein the nucleotide sequence of the nucleic acid molecule encodes at least one AAV Rep polypeptide, wherein the Rep polypeptide-encoding sequence has two or three of the following features:
(i) it is not operably-associated with a functional AAV p5 promoter;
(ii) it does not comprise a functional AAV p19 promoter or it does not encode functional Rep52 and Rep40 polypeptides;
(iii) it does not comprise a functional AAV p40 promoter or it does not comprise a functional adenovirus inhibitor sequence.

Preferably, the nucleotide sequence encodes functional AAV Rep78 and Rep68 polypeptides. Preferably, the nucleic acid molecule, in the absence of an operably-associated promoter, is not capable of expressing functional AAV Rep52 or Rep40 polypeptides.

The nucleic acid molecule of the invention may be DNA or RNA. It may be single-stranded or double-stranded.

As used herein, the term “rep gene” refers to a gene that encodes one or more open reading frames (ORFs), wherein each of said ORFs encodes an AAV Rep non-structural protein, or variant or derivative thereof. These AAV Rep non-structural proteins (or variants or derivatives thereof) are involved in AAV genome replication and/or AAV genome packaging.

The wild-type rep gene comprises three promoters: p5, p19 and p40. Two overlapping messenger ribonucleic acids (mRNAs) of different lengths can be produced from p5 and from p19. Each of these mRNAs contains an intron which can be either spliced out or not using a single splice donor site and two different splice acceptor sites. Thus, six different mRNAs can be formed, of which only four are functional. The two mRNAs that fail to remove the intron (one transcribed from p5 and one from p19) read through to a shared terminator sequence and encode Rep78 and Rep52, respectively. Removal of the intron and use of the 5′-most splice acceptor site does not result in production of any functional Rep protein—it cannot produce the correct Rep68 or Rep40 proteins as the frame of the remainder of the sequence is shifted, and it will also not produce the correct C-terminus of Rep78 or Rep52 because their terminator is spliced out. Conversely, removal of the intron and use of the 3′ splice acceptor will include the correct C-terminus for Rep68 and Rep40, whilst splicing out the terminator of Rep78 and Rep52. Hence the only functional splicing either avoids splicing out the intron altogether (producing Rep78 and Rep52) or uses the 3′ splice acceptor (to produce Rep68 and Rep40). Consequently, four different functional Rep proteins with overlapping sequences can be synthesized from these promoters.

In the wild-type rep gene, the p40 promoter is located at the 3′ end. Transcription of the Cap proteins (VP1, VP2 and VP3) is initiated from this promoter in the wild-type AAV genome.

The four wild-type Rep proteins are Rep78, Rep68, Rep52 and Rep40. Hence the wild-type rep gene is one which encodes the four Rep proteins Rep78, Rep68, Rep52 and Rep40.

As used herein, the term “rep gene” includes wild-type rep genes and derivatives thereof; and artificial rep genes which have equivalent functions. The wild-type rep gene encodes functional Rep78, Rep68, Rep52 and Rep40 polypeptides.

In a particularly-preferred embodiment, the nucleotide sequence encodes functional Rep78 and Rep68 polypeptides.

As used herein, the term Rep78 polypeptide refers to a polypeptide of SEQ ID NO: 22 or variant thereof having a least 80%, 85%, 90%, 95% or 99% sequence identify thereto and which encodes a functional Rep78 polypeptide. As used herein, the term Rep68 polypeptide refers to a polypeptide of SEQ ID NO: 23 or variant thereof having a least 80%, 85%, 90%, 95% or 99% sequence identify thereto and which encodes a functional Rep68 polypeptide.

In the production of AAVs, in the absence of sufficient functional Rep polypeptides, lower titres (e.g. genome copies) would be observed (which could be determined by qPCR), due to the fact that there is less ITR plasmid to be packaged and that it would not be effectively packaged. The observation might also include an exaggerated empty:full particle ratio; this could be determined by ELISA or optical density measurement.

The Rep 78/68 polypeptides bind ATP and have helicase activity and may be involved in assisting with the accumulation of single-stranded genome pre-cursors and assisting in the packaging of newly-formed DNA strands into preformed AAV capsid.

It is possible to determine whether or not a Rep 78 or Rep 68 polypeptide variant is being expressed by Western blot to determine whether these polypeptides are being produced at the correct molecular weight.

The functionality of these polypeptides may be determined in their purified form using a bioluminescent ATP assay that determine the consumption of ATP. As both Rep78 and Rep68 have helicase activity, an appropriate helicase assay may also be used.

A test Rep78 polypeptide having a level of ATP consumption in a bioluminescent ATP assay which is at least 80% (preferably at least 90%) of the consumption level of a wild-type Rep78 polypeptide (e.g. of SEQ ID NO: 22) may be considered to be a functional Rep78 polypeptide.

A test Rep68 polypeptide having a level of ATP consumption in a bioluminescent ATP assay which is at least 80% (preferably at least 90%) of the consumption level of a wild-type Rep68 polypeptide (e.g. of SEQ ID NO: 23) may be considered to be a functional Rep68 polypeptide.

A test Rep78 polypeptide having a level of helicase activity which is at least 80% (preferably at least 90%) of the activity level of a wild-type Rep78 polypeptide (e.g. of SEQ ID NO: 22) may be considered to be a functional Rep78 polypeptide.

A test Rep68 polypeptide having a level of helicase activity which is at least 80% (preferably at least 90%) of the activity level of a wild-type Rep68 polypeptide (e.g. of SEQ ID NO: 23) may be considered to be a functional Rep68 polypeptide.

The wild-type AAV (serotype 2) rep gene nucleotide sequence is given in SEQ ID NO: 1.

In one embodiment, the term “rep gene” or Rep polypeptide-encoding sequence refers to a nucleotide sequence having at least 70%, 80%, 85%, 90%, 95%, 99% or 100% sequence identity to SEQ ID NO: 1 and which encodes one or more Rep78, Rep68, Rep52 and Rep40 polypeptides, preferably functional Rep78 and 68 polypeptides.

The Rep52 and Rep40 nucleotide sequences are given herein in SEQ ID NOs: 16 and 17. The Rep78 and Rep68 nucleotide sequences are given herein in SEQ ID NOs: 20 and 21.

As used herein, the term “cap gene” refers to a gene that encodes one or more open reading frames (ORFs), wherein each of said ORFs encodes an AAV Cap structural protein, or variant or derivative thereof. These AAV Cap structural proteins (or variants or derivatives thereof) form the AAV capsid.

The three Cap proteins must function to enable the production of an infectious AAV virus particle which is capable of infecting a suitable cell. The three Cap proteins are VP1, VP2 and VP3, which are generally 87 kDa, 72 kDa and 62 kDa in size, respectively. Hence the cap gene is one which encodes the three Cap proteins VP1, VP2 and VP3.

In the wild-type AAV, these three proteins are translated from the p40 promoter to form a single mRNA. After this mRNA is synthesized, either a long or a short intron can be excised, resulting in the formation of a 2.3 kb or a 2.6 kb mRNA.

The AAV capsid is composed of 60 capsid protein subunits (VP1, VP2, and VP3) that are arranged in an icosahedral symmetry in a ratio of 1:1:10, with an estimated size of 3.9 MDa.

As used herein, the term “cap gene” includes wild-type cap genes and derivatives thereof, and artificial cap genes which have equivalent functions. The AAV (serotype 2) cap gene nucleotide sequence and Cap polypeptide sequences are given in SEQ ID NOs: 2 and 3, respectively. As used herein, the term “cap gene” refers preferably to a nucleotide sequence having the sequence given in SEQ ID NO: 2 or a nucleotide sequence encoding a polypeptide of SEQ ID NO: 3; or a nucleotide sequence having at least 70%, 80%, 85% 90%, 95% or 99% sequence identity to SEQ ID NO: 2 or at least 80%, 90%, 95% or 99% nucleotide sequence identity to a nucleotide sequence encoding a polypeptide of SEQ ID NO: 3, and which encodes VP1, VP2 and VP3 polypeptides.

The rep and cap genes are preferably viral genes or derived from viral genes. More preferably, they are AAV genes or derived from AAV genes. In some embodiments, the AAV is an Adeno-associated dependoparvovirus A. In other embodiments, the AAV is an Adeno-associated dependoparvovirus B.

11 different AAV serotypes are known. All of the known serotypes can infect cells from multiple diverse tissue types. Tissue specificity is determined by the capsid serotype. The AAV may be from serotype 1, 2, 3, 4, 5, 6, 7, 8, 9, 10 or 11. Preferably, the AAV is serotype 1, 2, 5, 6, 7, 8 or 9. Most preferably, the AAV serotype is 5 (i.e. AAV5).

The rep and cap genes (and each of the protein-encoding ORFs therein) may be from one or more different viruses (e.g. 2, 3 or 4 different viruses). For example, the rep gene may be from AAV2, whilst the cap gene may be from AAV5.

It is recognised by those in the art that the rep and cap genes of AAV vary by clade and isolate. The sequences of these genes from all such clades and isolates are encompassed herein, as well as derivatives thereof.

As used herein, the term “recombinant AAV genome” refers to an AAV genome comprising a transgene (in place of the rep and cap genes) flanked by AAV inverted terminal repeats (ITRs). As used herein, the terms “AAV genome”, “AAV Transfer vector” and “Transfer Plasmid” are used interchangeably herein. They all refer to a vector comprising 5′- and 3′-viral (preferably AAV) inverted terminal repeats (ITRs) flanking a transgene.

The transgene may be a coding or non-coding sequence. It may be genomic DNA or cDNA. Preferably, the transgene encodes a polypeptide or a fragment thereof. Preferably, the transgene is operably associated with one or more transcriptional and/or translational control elements (e.g. an enhancer, promoter, terminator sequence, etc.).

In some embodiments, the transgene codes for a therapeutic polypeptide or a fragment thereof. Examples of preferred therapeutic polypeptides include antibodies, CAR-T molecules, scFV, BiTEs, DARPins and T-cell receptors.

In some embodiments, the therapeutic polypeptide is a G-protein coupled receptor (GPCR), e.g. DRD1. In some embodiments, the therapeutic polypeptide is an immunotherapy target, e.g. CD19, CD40 or CD38. In some embodiments, the therapeutic polypeptide is a functioning copy of a gene involved in human vision or retinal function, e.g. RPE65 or REP. In some embodiments, the therapeutic polypeptide is a functioning copy of a gene involved in human blood production or is a blood component, e.g. Factor IX, or those involved in beta and alpha thalassemia or sickle cell anaemia. In some embodiments, the therapeutic polypeptide is a functioning copy of a gene involved in immune function such as that in severe combined immune-deficiency (SCID) or Adenosine deaminase deficiency (ADA-SCID). In some embodiments, the therapeutic polypeptide is a protein which increases/decreases proliferation of cells, e.g. a growth factor receptor. In some embodiments, the therapeutic polypeptide is an ion channel polypeptide.

In some preferred embodiments, the therapeutic polypeptide is an immune checkpoint molecule. Preferably, the immune checkpoint molecule is a member of the tumour necrosis factor (TNF) receptor superfamily (e.g. CD27, CD40, OX40, GITR or CD137) or a member of the B7-CD28 superfamily (e.g. CD28, CTLA4 or ICOS). Preferably, the immune checkpoint molecule is PD1, PDL1, CTLA4, Lag1 or GITR.

In some preferred embodiments, the transgene encodes a CRISPR enzyme (e.g. Cas9, dCas9, Cpf1 or a variant or derivative thereof) or a CRISPR sgRNA.

The wild-type AAV p5 promoter promotes expression of Rep 78 and Rep 68 polypeptides. The p5 promoter is located at the 5′ end of the wild-type rep gene.

The wild-type AAV2 p5 promoter has the nucleotide sequence as given in SEQ ID NO: 4. The core sequence is highlighted in bold.

As used herein, the term “functional p5 promoter” refers to a nucleotide sequence which consists of or comprises the nucleotide sequence of SEQ ID NO: 4 or a variant thereof having at least 80%, 85%, 90%, 95% or 99% sequence identity thereto and which is capable of promoting the transcription of an operably-associated nucleotide molecule which encodes one or more AAV Rep polypeptides, preferably the Rep 78 and Rep68 polypeptides.

The level of activity of a p5 promoter may be determined by operably-associating a test p5 promoter sequence with a suitable transgene and assaying for the level of expression of the transgene.

A level of expression which is less than 5% (preferably less than 1%) of the expression level from a wild-type p5 promoter when operably associated with the same transgene may be considered to be not functional.

In some embodiments, in the nucleic acid molecule of the invention, (i) the Rep polypeptide-encoding sequence is not operably-associated with a functional AAV p5 promoter. Preferably, this nucleotide sequence is located upstream (5′) of the Rep polypeptide-encoding sequence, more preferably immediately upstream of the Rep polypeptide-encoding sequence (i.e. contiguously-linked to the 5′-end of the Rep polypeptide-encoding sequence). In this way, expression of Rep78 and Rep68 polypeptide is reduced, thus reducing the toxicity of these polypeptides to an adenovirus.

In the absence of an operably-associated promoter, the Rep 78 and/or Rep 68 polypeptides are only capable of being expressed from the nucleic acid molecule of the invention at a low, baseline or minimal level.

For example, the wild-type AAV p5 promoter sequence (e.g. SEQ ID NO: 4) might be rendered non-functional by the presence of a mutation in the core region (as highlighted above) or it might have a mutation in the promoter's TATA element, whereby the TATA element cannot be bound by the TATA-binding protein and/or other transcription factors which are needed in order to initiate transcription.

In one embodiment, therefore, the Rep polypeptide-encoding sequence is operably-associated with an AAV p5 promoter which has one or more mutations in the core region and/or in the TATA element. Preferably, these mutations reduce the promoter activity of the AAV p5 promoter compared to a promoter of SEQ ID NO: 4, most preferably to render it not functional (as defined above).

In some preferred embodiments, the Rep polypeptide-encoding sequence is operably-associated with a nucleotide sequence which consists of or comprises a variant of the nucleotide sequence of SEQ ID NO: 4 having at least 80%, 85%, 90%, 95% or 99% sequence identity thereto and wherein, if the variant is operably-associated with a transgene, the expression level of the transgene is less than 5% (preferably less than 1%) of the expression level from a promoter having SEQ ID NO: 4 when operably-associated with the transgene.

In other embodiments, the Rep polypeptide-encoding sequence is not operably-associated with a functional or a non-functional AAV p5 promoter.

In this embodiment, the Rep polypeptide-encoding sequence may be operably-associated with a nucleotide sequence which has less than 99%, 95%, 90% or 85% sequence identity to SEQ ID NO: 4, and which preferably has no or essentially no promoter activity.

In some embodiments, the Rep polypeptide-encoding sequence is not operably-associated with an IRES element. In some embodiments, the p5 promoter is not replaced by an IRES.

In some embodiments of this aspect of the invention, the Rep polypeptide-encoding sequence is operably-associated with SEQ ID NO: 5 (a sequence that forms part of the 5′-untranslated region (UTR) of the human beta-globin gene):

The wild-type AAV p19 promoter promotes expression of Rep 52 and Rep 40 polypeptides. The p19 promoter is located within the wild-type rep gene.

The wild-type AAV2 p19 promoter has the nucleotide sequence as given in SEQ ID NO: 6. The highlighted sections are the TATA box and the TSS element.

As used herein, the term “functional p19 promoter” refers to a nucleotide sequence which consists of or comprises the nucleotide sequence of SEQ ID NO: 6 or a variant thereof having at least 80%, 85%, 90%, 95% or 99% sequence identity thereto and which is capable of promoting the transcription of an operably-associated nucleotide molecule which encodes one or more AAV Rep polypeptides, preferably the Rep 52 and Rep40 polypeptides.

The level of activity of the p19 promoter may be determined by operably-associating a test p19 promoter sequence with a suitable transgene and assaying for the level of expression of the transgene. A level of expression which is less than 5% (preferably less than 1%) of the expression level from a wild-type p19 promoter (when operably associated with the same transgene) may be considered to be not functional.

In some embodiments, in the nucleic acid molecule of the invention, (ii) the Rep polypeptide-encoding sequence does not comprise a functional AAV p19 promoter. In the absence of an operably-associated promoter, the Rep 52 and/or Rep 40 polypeptides are not capable of being expressed from the nucleic acid molecule of the invention. In this way, expression of Rep52 and/or Rep40 polypeptides is prevented or inhibited. These polypeptides are not essential for production of recombinant AAV particles, but they are capable of inhibiting adenoviral production. Consequently, the prevention or inhibition of the expression of Rep52 and/or Rep40 polypeptides enhances adenoviral production in systems wherein an adenovirus comprises a nucleic acid molecule of the invention.

For example, the wild-type AAV p19 promoter sequence (e.g. SEQ ID NO: 6) might be rendered non-functional by the presence of a mutation in the TSS element or it might have a mutation in the promoter's TATA element, whereby the TATA element cannot be bound by the TATA-binding protein and/or other transcription factors which are needed in order to initiate transcription.

In one embodiment, therefore, the Rep polypeptide-encoding sequence is operably-associated with an AAV p19 promoter which has one or more mutations in the TSS element and/or in the TATA element. Preferably, these mutations reduce the promoter activity of the AAV p19 promoter compared to a promoter of SEQ ID NO: 6, most preferably to render it not functional (as defined above).

In some embodiments, the AAV p19 promoter has a deletion which comprises some or all of the TATA box. More preferably, the Rep polypeptide-encoding sequence has a p19 promoter wherein the TATA box has been ablated by substituting one or more (e.g. 1, 2, 3 or 4) of the nucleotides of the TATA box for an alternative nucleotide, for example converting the TATA sequence to TTTT. Preferably, the change is a synonymous mutation or the change preserves the frame of the coding sequence. In these embodiments, the p19 promoter may still be functional, or non-functional.

One example of a non-functional p19 sequence is given in SEQ ID NO: 7. The highlighted sequence is a mutated TATA box element which makes the promoter non-functional.

In some preferred embodiments, the Rep polypeptide-encoding sequence is operably-associated with a nucleotide sequence which consists of or comprises a variant of the nucleotide sequence of SEQ ID NO: 6 having at least 80%, 85%, 90%, 95% or 99% sequence identity thereto and wherein, if the variant is operably-associated with a transgene, the expression level of the transgene is less than 5% (preferably less than 1%) of the expression level from a promoter having SEQ ID NO: 6 when operably-associated with the transgene.

In other embodiments, the Rep-encoding polypeptide sequence encodes a functional AAV p19 promoter but it does not encode functional Rep 52 and/or Rep40 polypeptides. Preferably, the activity of the Rep78/68 polypeptides is not significantly affected.

For example, the start codon for the Rep52/40 polypeptides may be mutated to eliminate expression of these polypeptides. In one example, the nucleotide sequence encoding the start codon (methionine) may be mutated, preferably without significantly impacting the expression of the Rep78/68 polypeptides.

Examples of non-functional Rep52 sequences include a nucleotide sequence which consists of or comprises a variant of the nucleotide sequence of SEQ ID NO: 16 or a variant of a nucleotide sequence which encodes the amino acid sequence of SEQ ID NO: 18, which has at least 80%, 85%, 90%, 95% or 99% sequence identity thereto and which does not encode a functional Rep52 polypeptide sequence.

Examples of non-functional Rep40 sequences include a nucleotide sequence which consists of or comprises a variant of the nucleotide sequence of SEQ ID NO: 17 or a variant of a nucleotide sequence which encodes the amino acid sequence of SEQ ID NO: 19, which has at least 80%, 85%, 90%, 95% or 99% sequence identity thereto and which does not encode a functional Rep40 polypeptide sequence.

The Rep 52/40 proteins bind ATP and have helicase activity and may be involved in assisting with the accumulation of single-stranded genome pre-cursors and assisting in the packaging of newly-formed DNA strands into preformed AAV capsid. They are often considered disposable for AAV production, unlike Rep78 and Rep68.

It is possible to determine whether or not a Rep 52 or Rep 40 polypeptide variant is being expressed by Western blot to determine whether these polypeptides are being produced at the correct molecular weight. The functionality of these polypeptides may be determined in their purified form using a bioluminescent ATP assay that determine the consumption of ATP. As both Rep52 and Rep40 have helicase activity, an appropriate helicase assay may also be used.

A test Rep52 polypeptide having a level of helicase activity which is less than 5% (preferably less than 1%) of the activity level of a wild-type Rep52 polypeptide (e.g. of SEQ ID NO: 18) may be considered to be a non-functional Rep52 polypeptide.

A test Rep40 polypeptide having a level of helicase activity which is less than 5% (preferably less than 1%) of the activity level of a wild-type Rep40 polypeptide (e.g. of SEQ ID NO: 19) may be considered to be a non-functional Rep40 polypeptide.

The wild-type AAV p40 promoter promotes expression of the AAV Cap polypeptides. The p40 promoter is located near the 3′ end of the wild-type AAV rep gene.

The wild-type AAV2 p40 promoter has the nucleotide sequence given in SEQ ID NO: 8. The highlighted element is the TATA element.

As used herein, the term “functional p40 promoter” refers to a nucleotide sequence which consists of or comprises the nucleotide sequence of SEQ ID NO: 8 or a variant thereof having at least 80%, 85%, 90%, 95% or 99% sequence identity thereto and which is capable of promoting the transcription of an operably-associated nucleotide molecule which encodes one or more AAV Rep polypeptides, preferably one or more of the AAV Cap polypeptides.

The level of activity of a p40 promoter may be determined by operably-associating a test p40 promoter sequence with a suitable transgene and assaying for the level of expression of the transgene. A level of expression which is less than 5% (preferably less than 1%) of the expression level from a wild-type p40 promoter (when operably-associated with the same transgene) may be considered to be not functional.

In some embodiments, in the nucleic acid molecule of the invention, (ii) the Rep polypeptide-encoding sequence does not comprise a functional AAV p40 promoter. In this way, the adenovirus inhibitor sequence is not transcribed.

For example, the wild-type AAV p40 promoter sequence (e.g. SEQ ID NO: 8) might be rendered non-functional by the presence of a mutation in the promoter's TATA element, whereby the TATA element cannot be bound by the TATA-binding protein and/or other transcription factors which are needed in order to initiate transcription.

In one embodiment, therefore, the Rep polypeptide-encoding sequence is operably-associated with an AAV p40 promoter which has one or more mutations in the TATA element. Preferably, these mutations reduce the promoter activity of the AAV p40 promoter compared to a promoter of SEQ ID NO: 8, most preferably to render it not functional (as defined above).

In some preferred embodiments, the Rep polypeptide-encoding sequence is operably-associated with a nucleotide sequence which consists of or comprises a variant of the nucleotide sequence of SEQ ID NO: 8 having at least 80%, 85%, 90%, 95% or 99% sequence identity thereto and wherein, when the variant is operably-associated with a transgene, the expression level of the transgene is less than 5% (preferably less than 1%) of the expression level from a promoter having SEQ ID NO: 8 when operably-associated with the transgene.

In other embodiments, the Rep polypeptide-encoding sequence is not operably-associated with a functional or a non-functional AAV p40 promoter. In this embodiment, the Rep polypeptide-encoding sequence may be operably-associated with a nucleotide sequence which has less than 99%, 95%, 90% or 85% sequence identity to SEQ ID NO: 8, and which preferably has no or essentially no promoter activity.

A preferred non-functional p40 promoter sequence is given in SEQ ID NO: 9. In this sequence, the TATA box element, transcriptional start site, and transcription factor binding sites are mutated, while the Rep78 and Rep68 polypeptide coding sequences are maintained.

The wild-type AAV p40 promoter sequence comprises an adenovirus inhibitor sequence. As used herein, the term “functional adenovirus inhibitor sequence” refers to a nucleotide sequence wherein when it is present in cis of the adenovirus genome, it leads to significant inhibition of replication of the adenovirus. The wild-type AAV2 adenovirus inhibitor has the sequence in SEQ ID NO: 10. This sequence forms the p40 promoter and adenovirus inhibitor sequence. The TATA element and transcriptional start site form the core of the inhibitor sequence.

Preferably, a functional adenovirus inhibitor sequence is defined as one which has the sequence shown in SEQ ID NO: 10, or a variant thereof which has at least 80%, 85%, 90% or 95% sequence identity thereto and which is capable of inhibiting adenoviral vector replication in a host cell.

The level of activity of the adenovirus inhibitor sequence may be determined by including an adenovirus inhibitor sequence (in cis or trans) into the sequence of an AV vector through molecular cloning and then attempting to recover the AV in mammalian cells. The insertion of a wild type adenovirus inhibitor sequence into an AV would completely prevent the recovery and outgrowth of any AV vector. By modifying the sequence of the adenovirus inhibitory sequence, it may be possible to recover AV vectors with varying degrees of success. This can be calculated by measuring the infectious titre of the recovered AV to determine the level of inhibition. Assays that can be used to measure AV titre include the TCID50 method and the plaque assay method.

A level of activity which is less than 5% (preferably less than 1%) of the activity level from a wild-type adenovirus inhibitor sequence (under the same conditions) may be considered to be not functional.

The wild-type AAV rep gene comprises a p40 promoter sequence which comprises an adenovirus inhibitor sequence.

In some embodiments, in the nucleic acid molecule of the invention, (iii) the Rep polypeptide-encoding sequence does not comprise a functional adenovirus inhibitor sequence.

For example, the adenovirus inhibitor sequence may be removed from the Rep polypeptide-encoding sequence or the adenovirus inhibitor sequence may be rendered non-functional (e.g. by making one or more mutations in the adenovirus inhibitor sequence).

The adenovirus inhibitor sequence may be modified in such a way that the transcription of the adenovirus inhibitor sequence, in a host cell, would not significantly inhibit the replication of a wild-type adenovirus in the host cell.

Preferably, the Rep polypeptide-encoding sequence encodes functional Rep78 and Rep68 polypeptides, i.e. the removal of the adenovirus inhibitor sequence or the making of the adenovirus inhibitor sequence non-functional does not affect the amino acid coding sequence of the Rep78 and Rep68 polypeptides.

Preferably, the AV inhibitor sequence within the p40 promoter is removed by synonymous codon exchange to maintain the Rep-coding amino acids. This reduces inhibition of the AV genes.

In some embodiments, it is not necessary to retain the correct coding sequences for the Rep52 and/or Rep40 polypeptides.

In one embodiment, the Rep polypeptide-encoding sequence comprises a functional p40 promoter sequence, i.e. the removal of the adenovirus inhibitor sequence or the making of the adenovirus inhibitor sequence non-functional does not affect, does not significantly reduce, or does not completely eliminate the function of the p40 promoter.

In another embodiment, the Rep polypeptide-encoding sequence does not comprise a functional p40 promoter sequence, i.e. the removal of the adenovirus inhibitor sequence or the making of the adenovirus inhibitor sequence non-functional completely eliminates the function of the p40 promoter.

Preferably, the nucleotide sequence without a functional p40 promoter sequence has the sequence given in SEQ ID NO: 11. In SEQ ID NO: 11, the AV inhibitor sequence within the p40 promoter has been removed by synonymous codon exchange to maintain the Rep coding amino acids, to reduce inhibition of the AV genes.

In some embodiments, the nucleic acid molecule of the invention does not comprise a heterologous promoter which is operably-associated with AAV Rep polypeptide-encoding sequence.

In other embodiments, the nucleic acid molecule of the invention does not comprise a promoter which is contiguous with AAV Rep polypeptide-encoding sequence.

As used herein, the term “operably-associated” in the context of a promoter and a gene means that the promoter and the gene in question are located within a distance from each other which is sufficiently close for the promoter to promote expression of the gene. In some embodiments, the promoter and the gene are juxtaposed or are contiguous.

Preferably, the Rep-polypeptide encoding sequence (rep gene) has the sequence given in SEQ ID NO: 12, or a variant thereof having at least 80%, 85%, 90 or 95% nucleotide sequence identity thereto, and wherein the p19 promoter is non-functional and the p40 promoter is non-functional and the adenoviral inhibitor sequence is non-functional. In SEQ ID NO: 12, the p19 and p40 promoters are ablated, retaining the Rep78 and Rep68 coding sequences.

In other preferred embodiments, the Rep polypeptide-encoding sequence has the features:

(i) it is not operably-associated with a functional AAV p5 promoter; and

(iii) it does not comprise a functional AAV p40 promoter and it does not comprise a functional adenovirus inhibitor sequence.

More preferably, the Rep polypeptide-encoding sequence has a p19 promoter wherein the TATA box has been ablated, e.g. by substituting one or more (e.g. 1, 2, 3 or 4) of the nucleotides of the TATA box for an alternative nucleotide, for example converting the TATA sequence to TTTT.

In some embodiments, the AAV p19 promoter has a deletion which comprises some or all of the TATA box, or one or more of the TATA bases have been changed from TATA to an alternative nucleotide. Preferably, the change is a synonymous mutation or the change preserves the frame of the coding sequence.

The p19 promoter may be functional, i.e. Rep40 and Rep52 polypeptides are capable of being expressed from it, or it may be non-functional.

In yet a further embodiment, the invention provides an adenoviral vector comprising a nucleic acid molecule of the invention. The nucleic acid molecule of the invention is stably integrated into the adenoviral genome.

In order to accommodate the nucleic acid molecule of the invention, part or all of one or more adenoviral genes may be deleted (preferably genes which are not adenoviral helper genes).

For example, a nucleic acid molecule of the invention may be inserted into one of the adenoviral Early genes or inserted in a site from which Early genes have been deleted from an adenovirus. In the latter example, the deleted Early genes may be trans-complimented by a cell line containing the deleted genes, e.g. HEK293 cells which contain the adenoviral E1A and E1B regions.

The nucleic acid molecule of the invention may be inserted into a region of an adenoviral genome containing an E1 deletion. In other instances, genes that are non-essential to the adenovirus can also be deleted and these sites can be used to insert a nucleic acid molecule of the invention. For example, a nucleic acid molecule of the invention may be inserted in the E3 region of an adenovirus because most E3 genes can be deleted in an adenoviral vector.

A nucleic acid molecule of the invention may be inserted into an adenoviral gene in sense or antisense orientation (with respect to the direction of transcription of the adenoviral gene). It is a preferred embodiment of the invention that the nucleic acid molecule of the invention will be in the same direction of transcription as the E4, E2A and E2B expression cassettes when it is inserted into the E1 region. This is to prevent the E1A promoter (that is often retained in E1-deleted AV's) from acting as a promoter to drive the rep gene expression. The E1A promoter cannot be removed because it contains the AV packaging signal.

It is a preferred embodiment of the invention that the Rep-coding sequence will not contain an upstream promoter.

In some preferred embodiments, a nucleic acid molecule of the invention is inserted into an adenoviral E1 gene, preferably wherein part of the E1 gene has been deleted. Preferably, the E1 gene is E1A and/or E1 B.

In one embodiment, the invention provides an adenoviral vector comprising a nucleic acid molecule of the invention, wherein the Rep polypeptide (encoded by the nucleic acid molecule of the invention) is expressed at a low, baseline or minimal level. Preferably, the Rep-polypeptide encoding sequence is not operably-associated with any functional promoter. As used herein, the term “low, baseline or minimal level” refers to a level of expression of the Rep78 polypeptide from the nucleic acid molecule of the invention which is less than 50%, 40%, 30%, 20% or 10% of the level of expression of a wild-type Rep 78 polypeptide which is operably-associated with a wild-type p5 promoter (in a wild-type AAV rep gene). In this way, sufficient Rep polypeptide is provided in order to enable the production of at least some AAV, but the level of Rep polypeptide expression is insufficient to completely inhibit adenovirus replication.

In some embodiments, the nucleic acid molecule of the invention is integrated into the adenoviral genome such that expression of the nucleic acid molecule of the invention is driven by an adenoviral promoter, preferably at a low or minimal level.

In other embodiments, the nucleic acid molecule of the invention is integrated into the adenoviral genome such that expression of the nucleic acid molecule of the invention is driven by an operably-associated heterologous promoter, preferably at a low or minimal level.

The operably-associated promoter may be a constitutive or inducible promoter. In some embodiments, the promoter is a constitutive promoter. In other embodiments, the promoter is inducible or repressible.

Examples of constitutive promoters include the CMV, SV40, PGK (human or mouse), HSV TK, SFFV, Ubiquitin, Elongation Factor Alpha, CHEF-1, FerH, Grp78, RSV, Adenovirus E1A, CAG or CMV-Beta-Globin promoter, or a promoter derived therefrom.

Preferably, the rep gene promoter is the SV40 promoter, or a promoter which is derived therefrom, or a promoter of equal or decreased strength compared to the SV40 promoter in human cells and human cell lines (e.g. HEK-293 cells).

In some embodiments, the promoter is inducible or repressible by the inclusion of an inducible or repressible regulatory (promoter) element.

For example, the promoter may be one which is inducible with doxycycline, tetracycline, IPTG or lactose.

In some embodiments of the invention, the nucleic acid molecule of the invention does not comprise any functional promoter and is not operably-associated with any functional promoter.

In some embodiments, the adenoviral vector of the invention additionally comprises a nucleotide molecule which encodes an AAV cap gene.

Preferably, the AAV cap gene is not juxtaposed with an AAV rep gene or a nucleic acid molecule of the invention. In particular, the AAV cap gene is preferably not operably-associated with a rep gene p40 promoter (either a wild-type AAV p40 promoter or a p40 promoter sequence of the invention).

For example, the AAV cap gene is preferably not present in the adenoviral genome within 1, 2, 3, 4 or 5 Kb of an AAV rep gene (or rep gene promoter) or a nucleic acid molecule of the invention.

Preferably, the AAV cap gene is operably-associated with a heterologous promoter. Preferably, the AAV cap gene is operably-associated with a constitutive or inducible promoter.

In some embodiments, the AAV cap gene is operably-associated with no promoter or a minimal promoter, e.g. the minimal promoter region of the CMV, RSV, SV40 promoters, or any composite core promoter region comprising a TATA box and sufficient regulatory binding sites to initiate basal transcription downstream of said TATA box. When no promoter is to be used, a TATA box will not be present. This maintains a low, baseline or minimal level of expression of the cap gene.

As used herein, the term “low, baseline or minimal level” refers to a level of expression of the cap gene which is less than 50%, 40%, 30%, 20% or 10% of the level of expression of a wild-type cap gene which is operably-associated with a wild-type p40 promoter (e.g. in a wild-type AAV).

As used herein, the term “heterologous” refers to a genetic element with which the gene in question is not naturally associated.

In a preferred embodiment of the invention, the AAV cap gene is not juxtaposed with an AAV rep gene or a nucleic acid molecule of the invention.

In particular, the AAV cap gene is integrated into an AV under the control of a promoter that is activated by a protein that is encoded in an AV encoding both an activator protein and a rep gene.

In yet a further embodiment of the invention, an adenoviral vector comprising a nucleic acid molecule of the invention (the first adenoviral vector) additionally encodes a polypeptide which is capable of transcriptionally-activating a (remote) promoter, for example a promoter which is present in a second adenoviral vector. Preferably, the promoter in the second adenoviral vector is one which is operably-associated with (i.e. drives expression of) an AAV cap gene.

In some embodiments, the adenoviral vector encodes a polypeptide which is capable of transcriptionally-activating a promoter which is not present in the adenoviral vector.

Examples of such polypeptides include the VP16 transcriptional activator from the herpes simplex virus and the transactivator domain from the p53 protein. These sequences may be linked to DNA binding domains such as those that bind the cumate binding site or the tetracycline binding site.

This allows transcription of the cap gene only to be induced when both first and second adenoviral vectors are present within the same cell, thereby reducing the burden of expressing the AAV cap gene during adenovirus manufacture.

The invention also provides a host cell comprising a nucleic acid molecule of the invention, located episomally within the host cell. The host cells may be isolated cells, e.g. they are not situated in a living animal or mammal. Preferably, the host cell is a mammalian cell.

Examples of mammalian cells include those from any organ or tissue from humans, mice, rats, hamsters, monkeys, rabbits, donkeys, horses, sheep, cows and apes. Preferably, the cells are human cells. The cells may be primary or immortalised cells. Preferred cells include HEK-293, HEK 293T, HEK-293E, HEK-293 FT, HEK-293S, HEK-293SG, HEK-293 FTM, HEK-293SGGD, HEK-293A, MDCK, C127, A549, HeLa, CHO, mouse myeloma, PerC6, 911 and Vero cell lines. HEK-293 cells have been modified to contain the E1A and E1 B proteins and this obviates the need for these proteins to be supplied on a Helper Plasmid. Similarly, PerC6 and 911 cells contain a similar modification and can also be used.

Most preferably, the human cells are HEK293, HEK293T, HEK293A, PerC6, 911 or HeLaRC32. Other preferred cells include Hela, CHO and VERO cells.

The inventors have discovered that the presence of a Rep-coding sequence and AAV ITRs sequences within the same adenovirus are detrimental to growth of the adenovirus. Whilst such an adenovirus can be recovered, its yield is typically 5-10 fold lower than when AV's contain each sequence independently. There are advantages to be obtained, therefore, by separating the Rep-coding sequence and AAV ITRs sequences, i.e. by placing them in two separate adenoviral vectors.

In yet another embodiment, the invention provides a kit comprising:

(A) a first adenoviral vector comprising a nucleic acid molecule of the invention and

(B) second adenoviral vector comprising
(i) a nucleic acid molecule encoding an AAV Cap polypeptide, and/or
(ii) a nucleic acid molecule encoding a recombinant AAV genome.

Preferably, the first adenoviral vector additionally encodes a polypeptide which is capable of transcriptionally-activating a promoter which is present in the second adenoviral vector. Preferably, the promoter in the second adenoviral vector is one which is operably-associated with (i.e. drives expression of) an AAV cap gene.

WO2019/020992 discloses that transcription of the Late adenoviral genes can be regulated (e.g. inhibited) by the insertion of a repressor element into the Major Late Promoter. By “switching off” expression of the adenoviral Late genes, the cell's protein-manufacturing capabilities can be diverted toward the production of a desired recombinant protein or AAV particles. Preferably, the adenoviral vector of this invention comprises a repressible Major Late Promoter (MLP), more preferably wherein the MLP comprises one or more repressor elements which are capable of regulating or controlling transcription of the adenoviral late genes, and wherein one or more of the repressor elements are inserted downstream of the MLP TATA box.

Preferred features for producing viral (preferably AAV) particles include the following:
wherein the one or more repressor elements are inserted between the MLP TATA box and the +1 position of transcription.
wherein the repressor element is one which is capable of being bound by a repressor protein.
wherein a gene encoding a repressor protein which is capable of binding to the repressor element is encoded within the adenoviral genome.
wherein the repressor protein is transcribed under the control of the MLP.
wherein the repressor protein is the tetracycline repressor, the lactose repressor or the ecdysone repressor, preferably the tetracycline repressor (TetR).
wherein the repressor element is a tetracycline repressor binding site comprising or consisting of the sequence set forth in SEQ ID NO: 13.
wherein the nucleotide sequence of the MLP comprises or consists of the sequence set forth in SEQ ID NO: 14 or 15.
wherein the presence of the repressor element does not affect production of the adenoviral E2B protein.
wherein the adenoviral vector encodes the adenovirus L4 100K protein and wherein the L4 100K protein is not under control of the MLP.
wherein a transgene is inserted within one of the adenoviral early regions, preferably within the adenoviral E1 region instead of in a Transfer Plasmid.
wherein the transgene comprises a Tripartite Leader (TPL) in its 5′-UTR.
wherein the transgene encodes a therapeutic polypeptide.
wherein the transgene encodes a virus protein, preferably a protein that is capable of assembly in or outside of a cell to produce a virus-like particle, preferably wherein the transgene encodes Norovirus VP1 or Hepatitis B HBsAG.

In yet a further embodiment, the invention also provides a process for producing a modified host cell, the process comprising the step:

(a) introducing a nucleic acid molecule of the invention into a host cell, such that the nucleic acid molecule becomes:
(i) stably integrated into the genome of the host cell, or
(ii) present episomally within the host cell.

Preferably, the nucleic acid molecule of the invention becomes present episomally within the host cell.

In some embodiments of the invention, the nucleic acid molecule of the invention in the cell does not comprise any functional promoter and it is not operably-associated with any functional promoter.

In some embodiments, the host cell is one which expresses or is capable of expressing a Cap polypeptide and/or AAV genome.

For example, the host cell may be one in which one or more DNA molecules comprising nucleotide sequences which encode the Cap polypeptide and/or AAV genome are stably integrated. The nucleotide sequences which encode Cap polypeptide and/or AAV genome are preferably operably-associated with suitable regulatory elements, e.g. inducible or constitutive promoters.

For example, the host cell may be one which comprises one or more DNA plasmids or vectors comprising nucleotide sequences which encode the Cap polypeptide and/or AAV genome. The nucleotide sequences which encode Cap polypeptide and/or AAV genome are preferably operably-associated with suitable regulatory elements, e.g. inducible or constitutive promoters.

The host cell may be an AAV packaging cell or an AAV producer cell.

As used herein, the term “introducing” one or more plasmids or vectors into a cell includes transformation, and any form of electroporation, conjugation, infection, transduction or transfection, inter alia.

In yet a further embodiment, the invention provides a process for producing a modified adenoviral vector, the process comprising the step of:

(a) introducing a nucleic acid molecule of the invention into an adenoviral vector.

Preferably, the nucleic acid molecule is stably integrated into the adenoviral vector genome.

In some embodiments of the invention, the nucleic acid molecule of the invention in the adenoviral vector genome does not comprise any functional promoter and it is not operably-associated with any functional promoter.

In yet a further embodiment, the invention provides a process for producing AAV particles, the process comprising the steps:

(a) infecting a mammalian host cell with a first adenoviral vector of the invention;

(b) infecting the host cell with a second adenoviral vector, wherein the second adenoviral vector comprises a recombinant AAV genome comprising a transgene, wherein at least one of the first and second adenoviral vectors comprise an AAV cap gene;

(c) culturing the mammalian host cell in a culture medium under conditions such that AAV particles comprising the transgene are produced; and

(d) isolating or purifying AAV particles from the cells or from the cell culture medium.

At least one of the adenoviral vectors also comprises sufficient helper genes for packaging the AAV genome (e.g. E4, E1, E2a and VA).

In yet a further embodiment, the invention provides a process for producing AAV particles, the process comprising the steps:

(a) infecting a mammalian host cell with an adenoviral vector, the vector comprising
(i) a recombinant AAV genome comprising a transgene, and
(ii) an AAV cap gene,

wherein the mammalian host cell comprises a nucleic acid molecule of the invention located episomally within the host cell or stably integrated into the host cell genome;

(b) culturing the mammalian host cell in a culture medium under conditions such that AAV particles comprising the transgene are produced; and

(c) isolating or purifying AAV particles from the host cells or from the cell culture medium.

The adenoviral vector also comprises sufficient helper genes for packaging the AAV genome (e.g. E4, E2a and VA, including an E2A gene).

In yet a further embodiment, the invention provides a process for producing AAV particles, the process comprising the steps:

(a) infecting a mammalian host cell with a first adenoviral vector of the invention, wherein the mammalian host cell comprises a recombinant AAV genome stably integrated into the host cell genome, wherein the recombinant AAV genome comprises a transgene, and wherein:

(i) the adenoviral vector additionally comprises an AAV cap gene, or

(ii) an AAV cap gene is stably integrated into the mammalian host cell genome, or

(iii) the cell is infected with a second adenoviral vector comprising an AAV cap gene;

(b) culturing the mammalian host cell in a culture medium under conditions such that AAV particles comprising the transgene are produced; and

(c) isolating or purifying AAV particles from the cells or from the cell culture medium.

At least one of the adenoviral vectors which are present in the cell also comprise sufficient helper genes for packaging the AAV genome (e.g. E4, E1A, E1 B and VA, and optionally an E2A gene).

In yet a further embodiment, the invention provides a process for producing recombinant AAV particles, the process comprising the steps:

(a) infecting a mammalian host cell with a first adenoviral vector of the invention, wherein:
(i) the first adenoviral vector additionally comprises an AAV cap gene, or
(ii) the cell is infected with a second adenoviral vector comprising an AAV cap gene;

(b) infecting the mammalian host cell with a recombinant AAV comprising a transgene;

(c) culturing the mammalian host cell in a culture medium under conditions such that AAV particles comprising the transgene are produced; and

(d) isolating and/or purifying AAV particles from the cells or from the cell culture medium.

At least one of the adenoviral vectors which are introduced (infected) into the cell also comprise sufficient helper genes for packaging the AAV genome (e.g. E4, E1A, E1 B and VA, and optionally an E2A gene).

The steps in Steps (a) and (b) may be carried out in any order. The initial recombinant AAV comprising a transgene may initially be made by any suitable means.

In the above embodiment, the initial recombinant AAV comprising a transgene and the adenoviral vector(s) may subsequently be reintroduced (e.g. reinfected) into further host cells in order to repeat the process. In instances where the major late promoter of the adenovirus is regulated, primarily AAV will be produced. Therefore, the process can be repeated by adding further adenoviral vectors to the host cells in addition to (e.g. a portion of) the AAV produced from the above embodiment, thereby passaging AAV through the addition of more adenoviral vector. As such, Steps (a) and/or (b) may be repeated, as necessary.

There are many established algorithms available to align two amino acid or nucleic acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid or nucleic acid sequences for comparison may be conducted, for example, by computer-implemented algorithms (e.g. GAP, BESTFIT, FASTA or TFASTA), or BLAST and BLAST 2.0 algorithms.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. (See Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters.

In other embodiments, a BLAST Global Alignment program may be used (such as that available from the NCBI) using a Needleman-Wunsch alignment of two protein sequences with the gap costs: Existence 11 and Extension 1.

The disclosure of each reference set forth herein is specifically incorporated herein by reference in its entirety.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

In the following Examples, references to “TERA vectors” are to adenovirus vectors wherein transcription of the late adenovirus genes from the major late promoter are regulated, as described in WO2019/020992. WO2019/020992 discloses that transcription of the Late adenoviral genes can be regulated (e.g. inhibited) by the insertion of a repressor element into the Major Late Promoter. By “switching off” expression of the adenoviral Late genes, the cell's protein-manufacturing capabilities can be diverted toward the production of a desired recombinant protein or AAV particles.

WO2019/020992 discloses that an adenoviral vector containing a repressor element in the Major Late Promoter can also encode the TetR protein downstream, and under the transcriptional control of the Major Late Promoter. In the absence of doxycycline, the TetR protein will bind to the Major Late Promoter repressor element and prevent the promoter's activity. In the presence of doxycycline, the TetR protein cannot bind to the repressor element in the Major Late Promoter. Consequently, in the presence of doxycycline, the Major Late Promoter of the adenovirus is active and the structural genes of the adenovirus are expressed and the virus can replicate. The vector “TERA-AAV” encodes the AAV transfer genome.

The vector “TERA-RepCap” encodes AAV Rep and Cap. In this construct, there are no functional Rep p5 or p40 promoters; no Rep adenovirus inhibitor sequence is present; and the Rep p19 promoter has been modified to delete the TATA box, although the promoter is still functional. The rep gene is inserted in the E1 region of the adenovirus. Cap is expressed from a CMV promoter. The cap gene is inserted into the E1 region of the adenovirus. The transcriptional orientation of the CMV promoter does not drive towards the Rep coding sequence.

The TERA2.0 approach refers to the process of adding TERA-AAV and TERA-RepCap to a cell.

The vectors TERA-RepCap5, TERA-RepCap6 and TERA-RepCap9 encode AAV Rep and the Cap gene from AAV5, AAV6 or AAV9, respectively. In TERA-RepCap5, the Cap gene is driven by a minimal CMV promoter region. In TERA-RepCap6 and TERA-RepCap9, the Cap gene is driven by the full length CMV promoter. In these constructs, there are no functional Rep p5 or p40 promoters; no Rep adenovirus inhibitor sequence is present; and the Rep p19 promoter has been modified to ablate the TATA box, although the promoter is still functional. The rep gene is inserted in the E1 region of the adenovirus. The Cap gene expression cassette is inserted into the E1 region of the adenovirus. The transcriptional orientation of the minimal CMV promoter or the full length CMV promoter does not drive towards the Rep coding sequence.

The triple plasmid transfection, or Helper-free, approach refers to the transfection of an adenoviral helper plasmid (as opposed to the use of an adenovirus), a plasmid encoding AAV Rep and Cap as they are found in nature, and a plasmid containing an AAV genome where a CMV driven EGFP expression cassette has been inserted between the AAV ITRs.

The plasmids pRepCap2, pRepCap5, pRepCap6, pRepCap9 encode AAV Rep and the Cap gene from AAV2, AAV5, AAV6 or AAV9, respectively. In these constructs the AAV rep and Cap genes are configured as they are found in nature.

The plasmid pAAV-EGFP encodes the left and right ITRs from an AAV genome. Inserted between the ITRs is a CMV promoter driving the expression of the EGFP reporter gene followed by a poly-adenylation signal.

The plasmid pHelper contains the adenoviral sequences required for AAV help. This includes the E4 region, the E2A region and the VA gene. This plasmid does not encode the adenovirus E1 region because this is found in the HEK293 cells that are used in the studies described herein.

### Example 1: AAV Rep Proteins Repress Transcription from the Adenovirus Major Late Promoter

To determine the effect of AAV Rep proteins on transcription from adenovirus major late promoter (MLP), the wildtype MLP or TetR repressible MLP sequence were inserted into plasmids expressing the EGFP reporter protein by molecular cloning methods. MLP promoter plasmids were co-transfected into HEK293 cells with the control CMV plasmid, or plasmid constructs expressing AAV Rep78, Rep68, Rep52, or Rep40 under control of the CMV promoter. EGFP reporter expression were quantified by flow cytometry 48 hours post transfection.

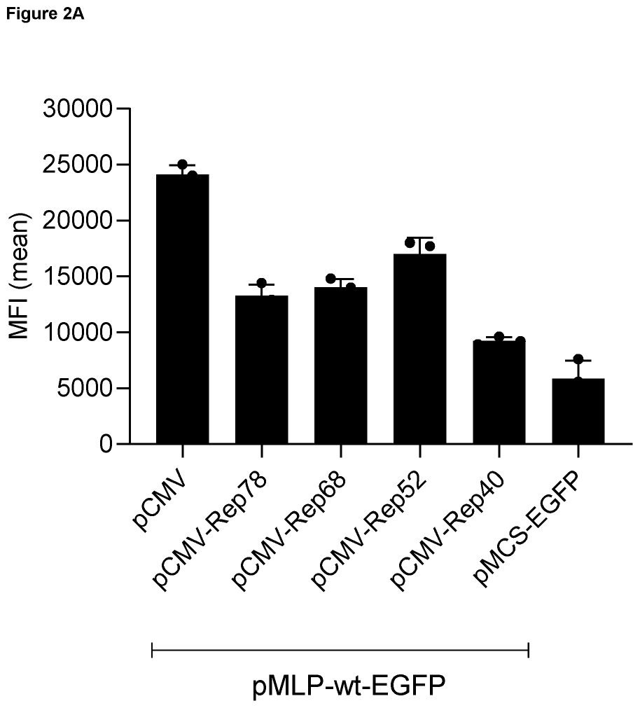

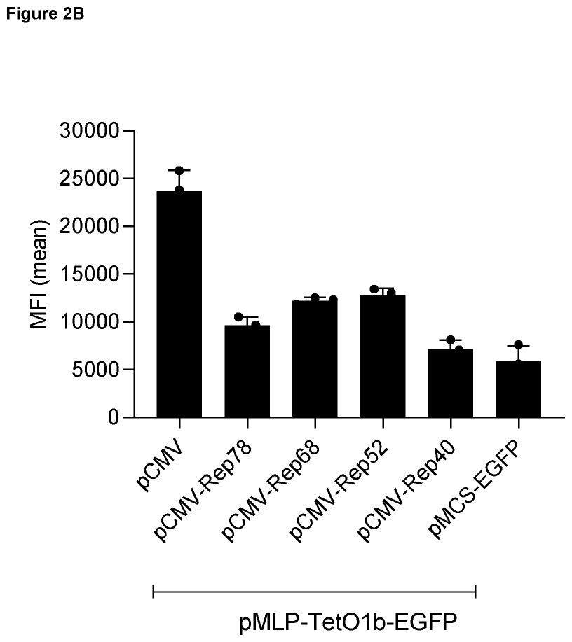

The results are shown in FIGS. 2A and 2B. These results show that high expression of the Rep proteins inhibit the activity of the adenoviral Major late Promoter or modified forms of the Major Late Promoter that contain a TetR binding site.

### Example 2: AAV Rep DNA is Stably Integrated and Replicated with the Adenovirus Genome

To construct an adenovirus vector encoding the AAV Rep78 and Rep68 polypeptides, the p5 and p19 promoters (involved in the transcription of AAV Rep52 and AAV Rep40) and the p40 adenovirus inhibitor sequence were scrambled (whilst maintaining the AAV Rep78 and Rep68 polypeptide) and inserted into the E1-deleted region of an adenoviral vector by molecular cloning methods.

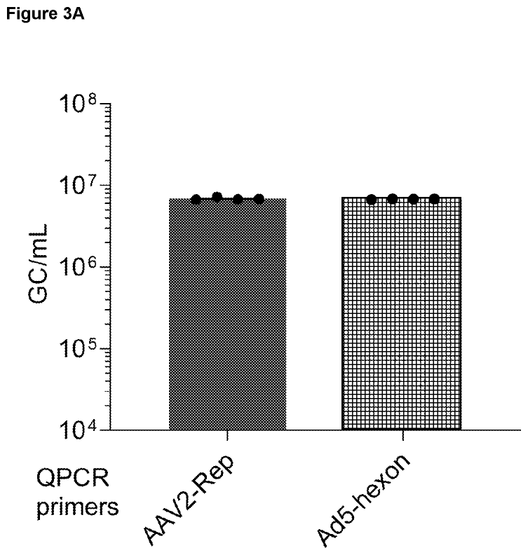

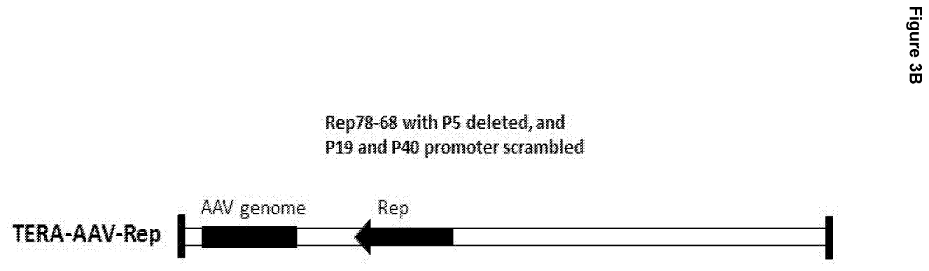

This resulted in the production of the vector shown in FIG. 3B where the adenoviral vector encodes the Rep coding sequence and also an AAV genome expressing EGFP.

The adenoviral vector genomes were transfected into HEK293 cells and viral vectors were harvested −15 days post transfection upon observation of full cytopathic effect. Vectors were subsequently passaged (>5) in HEK293 cells and adenoviral particles were treated with DNase to degrade any encapsulated DNA. Presence of the AAV Rep gene and adenovirus hexon gene sequence were determined by QPCR. The results are shown in FIG. 3A and the vector is shown in FIG. 3B.

FIG. 3A shows that the frequency of AAV Rep coding DNA and the adenoviral Hexon DNA are present in equal numbers, demonstrating that the Rep DNA insertion into the adenoviral genome is stable.

### Example 3: Co-Infection with TERA Vectors Significantly Increases the Production of AAV in HEK293 Cells Compare to Helper-Free Plasmid Transfection Method

HEK293 cells were seeded in a 48-well tissue culture plate format at 9e4 cells/well for 24 hours. Cells were triple transfected with the helper-free plasmids or co-infection of A) TERA encoding the AAV transfer genome with an EGFP expression cassette and the AAV2 Cap2 genes driven by the CMV promoter (TERA-AAV-Cap) at an MOI of 100, with B) TERA encoding the AAV transfer genome with an EGFP expression cassette and AAV Rep78-68s (TERA-AAV-Rep) at an MOI of 50, in the presence of doxycycline 0.5 ug/mL or DMSO. AAV vectors were harvested 96 hours post-transduction and encapsulated AAV particles and contaminating adenovirus particles were quantified by QPCR.

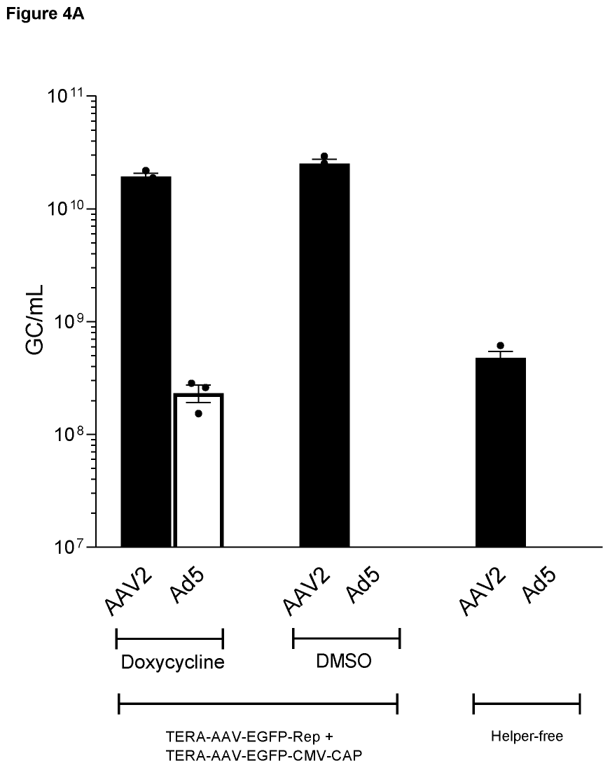

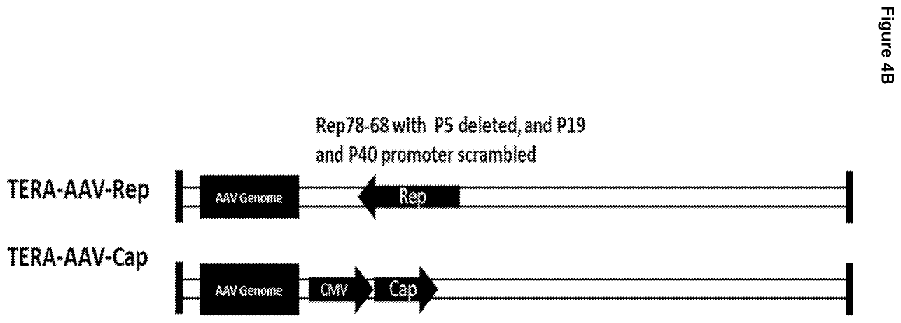

The results are shown in FIG. 4A and the vector in FIG. 4B. These results show that AAV can be produced at high titres using a combination of TERA-AAV-Cap and TERA-AAV-Rep. FIG. 4A shows that the titres achieved are considerably higher than those achieved with the triple transfection or helper-free approach. FIG. 4B shows a schematic representation of the adenoviral genomes containing AAV components for TERA-AAV-Rep and TERA-AAV-Cap.

### Example 4: Co-Infection with TERA Vectors Encoding the AAV Transfer Genome and AAV2 Cap with TERA Encoding AAV Rep78-68 Significantly Increases the Production of AAV in HEK293 Cells Compared to Helper-Free Plasmid Transfection Method

HEK293 cells were seeded in a 48-well tissue culture plate format at 9e4 cells/well for 24 hours. Cells were triple transfected with the helper-free plasmids or co-infection (at the indicated MOI) of A) TERA-AAV-Cap, with B) TERA-AAV-Rep, or C) TERA encoding the AAV Rep78-68s (TERA-Rep78-68), in the presence of doxycycline 0.5 ug/mL or DMSO. AAV vectors were harvested 96 hours post-transduction and encapsulated AAV particles and contaminating adenovirus particles were quantified by QPCR.

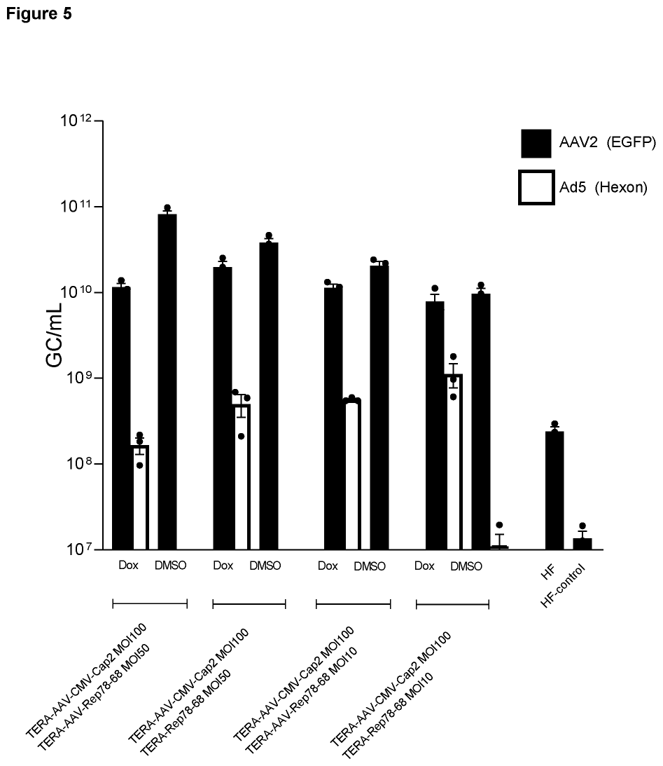

The results are shown in FIG. 5. These results show that two viruses that contain all of the AAV components provide improved AAV titres that are well above those achieved with the helper-free/triple plasmid transfection approach. A variety of viral vector combinations are shown in FIG. 5, demonstrating the flexibility and versatility of the approach. In each production run at least one viral vector contains the Rep construct of the invention.

### Example 5: Production of AAV2 Vectors in HEK293 Cells Using TERA2.0 System, Consisting of Two TERA Vectors, One Encoding the AAV Transfer Genome and the Second Encoding AAV Rep and Cap

Until the development of the invention described herein it was not possible to encode both AAV Rep and Cap into a single adenoviral vector. To simplify the approach to AAV manufacture, a new adenoviral vector was constructed encoding both AAV Rep and AAV Capsid from serotype 2 (TERA-RepCap). In this Example, AAV production using this new adenoviral vector (TERA-RepCap) in combination with TERA-AAV was tested, an approach termed TERA-2.0. This approach was compared to the triple plasmid transfection/helper-free method.

HEK293 cells were infected with TERA-AAV and TERA-RepCap to embody the TERA2.0 approach, each at an MOI of 25.

Additionally, HEK293 cells were triple transfected with the helper free plasmids (pAAV-EGFP; pRepCap2; pHelper). Cells were cultured for 4-days before vector harvest. Samples were treated with DNAse for 2 hours at 37° C. and quantified by qPCR using primers and probe against EGFP transgene. AAV vectors produced from these two approaches were used to infect fresh HEK293 cells and transducing units quantified by the TCID50 assay.

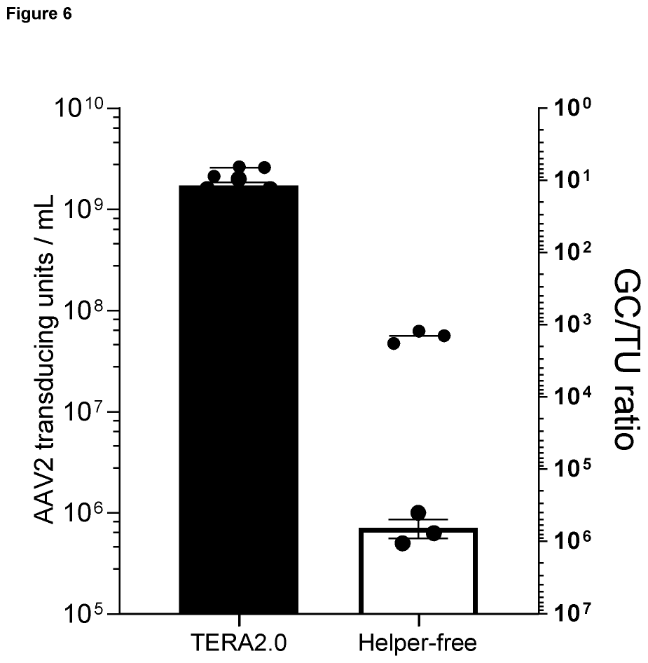

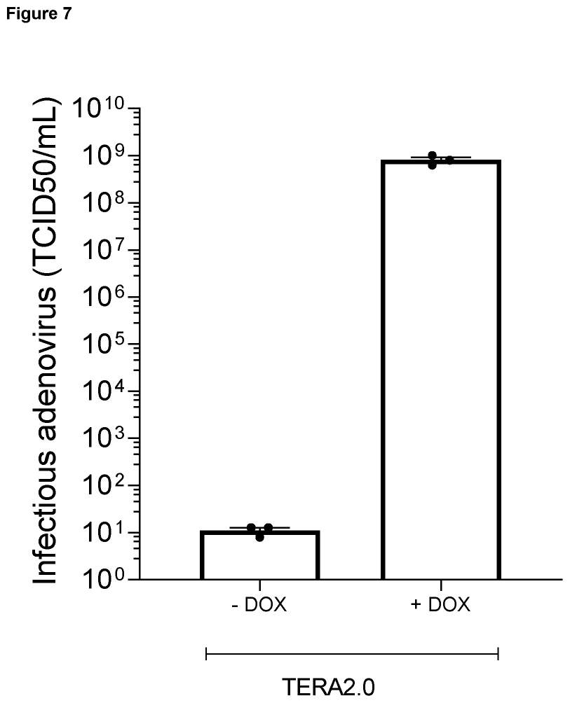

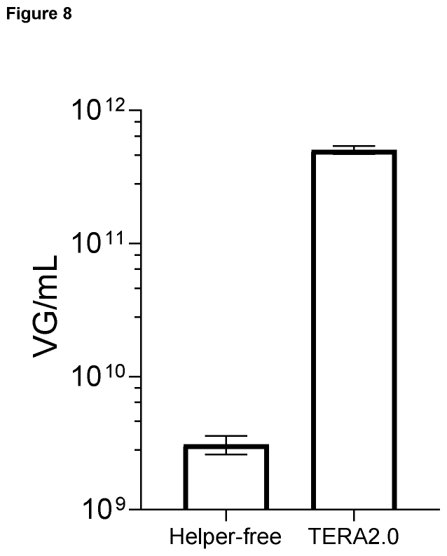

The results are shown in FIG. 6. Ratios of genome copies (GC) to transducing units (TU) shown on the right axis were determined by division of intact AAV particles determined by qPCR by transduction units measured from TCID50 assay. The results from this Example show that infection of cells using TERA2.0 (co-infection with TERA-AAV and TERA-RepCap), enabled a 1000-fold increase in transduction competent AAV vector production compared to preparations produced from triple plasmid transfection.

### Example 6: Determining the Levels of Contaminating Adenoviruses from an AAV2 Production Process Using the TERA2.0 Approach

AAV production was carried out in HEK293 cells which were infected using the TERA2.0 approach (co-infection with TERA-AAV and TERA-RepCap, each at an MOI of 25) and cultured for 4 days with doxycycline 0.5 ug/ml or DMSO (-DOX). In the absence of doxycycline (DMSO group) adenovirus late genes are repressed and virus replication cycle is truncated. Additionally, the presence of doxycycline in the growth media enables expression of late adenovirus genes from TERA vectors and the production of adenovirus. Vectors were harvested by three rounds of freeze-thaw and contaminating adenoviruses were detected by TCID50 assay by infecting fresh HEK293 cells, and cultured with doxycycline 0.5 ug/ml.

This Example shows that production of AAV vectors in the absence of doxycycline using two TERA vectors, one encoding the AAV transfer genome and a second encoding AAV Rep and Cap, significantly repressed replication of adenoviruses and produced a clean AAV preparation. Over 9 log reduction in contaminating adenovirus was observed compared to production of AAV in the presence of doxycycline.

### Example 7: Production of AAV2 Vectors in HEK293 Cells Using TERA2.0 System Compared to Triple Plasmid Transfection Method in 1 L Stir-Tank Bioreactor

Suspension HEK293 cells were infected using the TERA2.0 approach (co-infection with TERA-AAV and TERA-RepCap) each at an MOI of 25 TCID50 Units/cell.

Additionally, HEK293 cells were triple transfected with the helper free plasmids (pAAV-EGFP; pRepCap2; pHelper). Cells were cultured for 4-days before vector harvest. Samples were treated with DNAse for 2 hours at 37° C. and quantified by qPCR using primers and probe against the EGFP transgene to determine the titre of intact particles (viral genomes (VG)).

This Example shows that production of AAV2 vectors can be produced using co-infection of TERA vectors encoding an AAV transfer genome, and AAV Rep and Cap, in suspension-adapted HEK293 cells. The titre of AAV2 vectors achieved from this approach was over >100-fold increase in intact particles.

### Example 8: Assessment of Genetic Stability by qPCR of TERA Vector Encoding AAV Rep and Cap

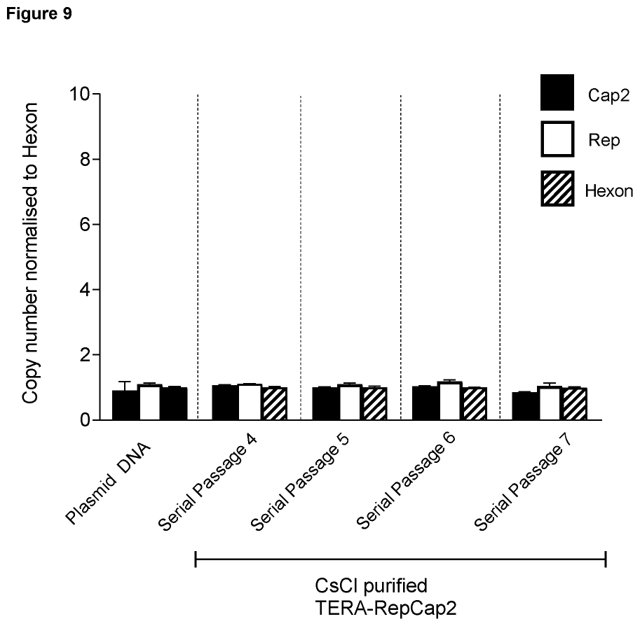

Plasmid DNA encoding TERA-RepCap was transfected into HEK293 cells for vector recovery and further amplified by serial passage. HEK293 cells were infected (MOI-15) in a HYPERFlask cell culture vessel. Vectors were harvested after 3 days and purified by caesium chloride ultracentrifugation to generate serial passage 4. This process was repeated to create vector passages 5, 6 and 7. DNA was extracted from purified TERA-RepCap vector at each passage and AAV2 rep and cap, and Ad5 hexon genes were quantified by qPCR. The data in FIG. 9 shows the copy number of AAV2 rep and cap genes relative to Ad5 hexon and compared to DNA plasmid encoding TERA-RepCap (ns, two-way ANOVA).

This Example shows that the AAV Rep and Cap genes are stably propagated within the TERA vector during serial passages in HEK293 cells.

### Example 9: Assessment of AAV Rep Expression from TERA Vector, Encoding AAV Rep and Cap. Western Blot Detection of Rep Proteins from the AAV Production Process Using TERA2.0 Approach

HEK293 cells were co-infected (MOI-25 each) with TERA vectors encoding the AAV transfer genome (TERA-AAV) and AAV Rep and Cap (TERA-RepCap). Samples were harvested 24 and 48 hours post-infection and total cellular extracts (25 uL) were probed with AAV2 Rep antibody by Western blot. Data presented as duplicate biological replicates.

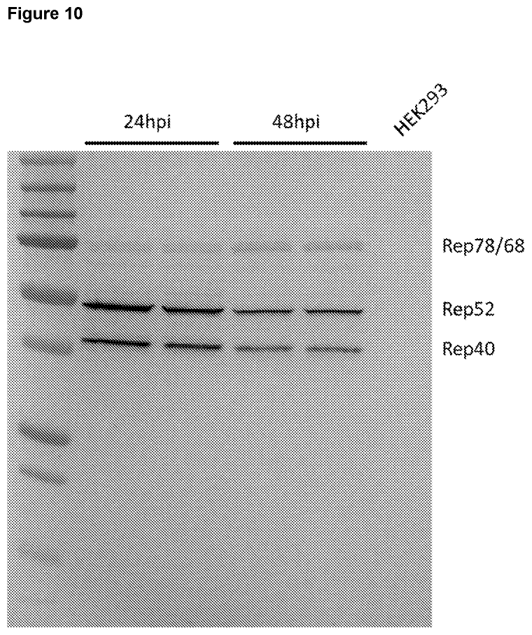

The results are shown in FIG. 10. This Example shows that high levels of AAV Rep 52 and 40 are expressed from the TERA vectors encoding AAV Rep and Cap and that lower levels of Rep78 and Rep68 are also expressed.

### Example 10: Assessment of AAV2 Capsid Proteins from Particles Produced Using TERA Vectors Encoding AAV Rep and Cap

HEK293 cells were co-infected (MOI-25 each) with TERA-AAV and TERE-RepCap (in presence or absence of doxycycline 0.5 μg/mL), or via co-infection of AAV2 particles (produced from TERA2.0) at 50 GC/cell with TERA-RepCap (MOI-25). Samples were harvested at 96 hours post infection and cell lysate (25 uL) was probed with anti-AAV2 VP1/2/3 antibody.

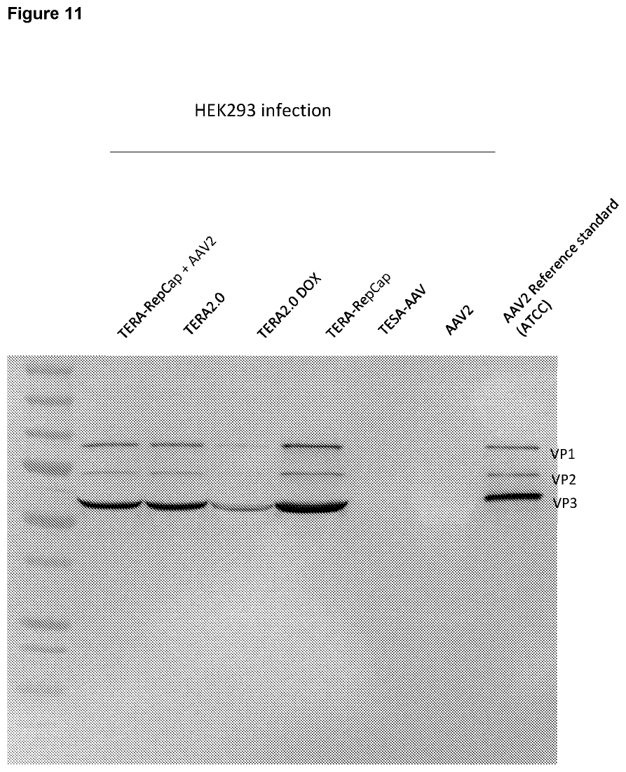

The results are shown in FIG. 11; an AAV2 standard (ATCC VR-1616) is also shown. This result shows that the relative composition of AAV2 capsid subunits VP1, VP2, and VP3 are identical to AAV2 vectors from the reference standard and also from material produced from the triple plasmid transfected method.

### Example 11: Production of AAV5 Vectors in HEK293 Cells Using TERA2.0 System, Consisting of Two TERA Vectors, One Encoding the AAV Transfer Genome and the Second Encoding AAV Rep and Cap5. AAV Production is Compared to Triple Plasmid Transfection Method

HEK293 cells were infected using the TERA2.0 approach described earlier, but where the AAV2 capsid was exchanged for the Capsid from AAV serotype 5 (TERA-AAV and TERA-RepCap5, wherein Rep is not express from a heterologous promoter and Cap5 is expressed from a minimal CMV promoter). TERA-AAV was used at an MOI of 25 and TERA-RepCap5 was used at 75 genome copies (GC) per cell. Alternatively, HEK293 cells were triple transfected with the helper free plasmids (pAAV-EGFP; pRepCap5; pHelper). Cells were cultured for 4-days before vector harvest. Samples were treated with DNAse for 2 hours at 37° C. and quantified by qPCR using primers and probes against the EGFP transgene.

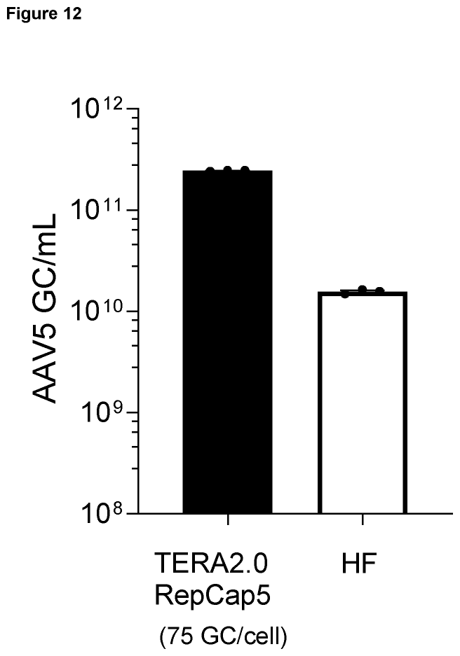

The results are shown in FIG. 12. The results from this Example shows that co-infection with two TERA vectors, encoding the AAV transfer genome and AAV rep and cap5 genes, enabled over a 16-fold increase in intact AAV5 particles compared to the triple plasmid transfection/helper-free approach.

### Example 12: Production of AAV6 Vectors in HEK293 Cells Using TERA2.0 System, Consisting of Two TERA Vectors, One Encoding the AAV Transfer Genome and the Second Encoding AAV Rep and Cap6. AAV Production is Compared to the Triple Plasmid Transfection Method

HEK293 Cells were infected using the TERA2.0 approach described earlier, but where the AAV2 capsid was exchanged for the Capsid from AAV serotype 6 (TERA-AAV and TERA-RepCap6). TERA-AAV was used at an MOI of 25 and TERA-RepCap6 was used at 75 genome copies (GC) per cell. Additionally, HEK 293 cells were triple transfected with the helper free plasmids (pAAV-EGFP; pRepCap6; pHelper). Cells were cultured for 4-days before vector harvest. Samples were treated with DNAse for 2 hours at 37° C. and quantified by qPCR using primers and probe against EGFP transgene.

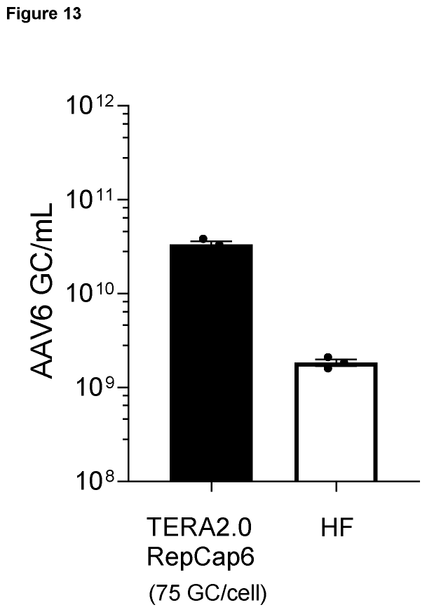

The results are shown in FIG. 13. The results from this Example show that co-infection with two TERA vectors, encoding the AAV transfer genome and AAV Rep and Cap6 genes, enabled over 18-fold increase in intact AAV6 particles compare to the triple plasmid transfection approach.

### Example 13: Production of AAV9 Vectors in HEK293 Cells Using TERA2.0 System, Consisting of Two TERA Vectors, One Encoding the AAV Transfer Genome and the Second Encoding AAV Rep and Cap9. AAV Production is Compared to Triple Plasmid Transfection Method

HEK293 Cells were infected using the TERA2.0 approach described earlier, but where the AAV2 capsid was exchanged for the Capsid from AAV serotype 9 (TERA-AAV and TERA-RepCap9). TERA-AAV was used at an MOI of 25 and TERA-RepCap9 was used at 50 genome copies (GC) per cell. Additionally, HEK 293 cells were triple transfected with the helper free plasmids (pAAV-EGFP; pRepCap9; pHelper). Cells were cultured for 4-days before vector harvest. Samples were treated with DNAse for 2 hours at 37° C. and quantified by qPCR using primers and probe against EGFP transgene

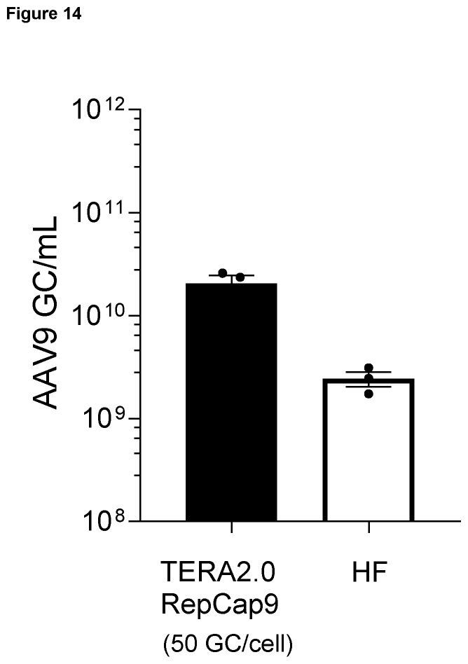

The results are shown in FIG. 14. These results show that co-infection with two TERA vectors, encoding the AAV transfer genome and AAV rep and Cap9 genes, enabled over 8.5-fold increase in intact AAV9 particles compared to the triple plasmid transfection approach.

### Example 14: Production of AAV2 Vectors in HEK293 Cells Using Two TERA Vectors I) Encoding AAV Rep (TERA-Rep) and II) Encoding AAV-EGFP and an AAV Cap2 Expression Cassette Driven from the CMV Promoter (TERA-AAV-EGFP-Cap2) Compared Against the Helper-Free Plasmid Method or Replacing TERA-Rep with a Plasmid Encoding the Rep Coding Sequence Driven by the Native AAV p5 Promoter

To produce AAV2-EGFP vectors, HEK293 cells were co-infected with the TERA-Rep (MOI5, 10, or 50) with TERA-AAV-EGFP-Cap2 (MOI100). This was compared to HEK293 cells transfected with plasmid p5-Rep, wherein the Rep78/68 polypeptide is expressed from its native p5 promoter and infected with TERA-AAV-EGFP-Cap2 (MOI100). Cells were cultured for 4-days before vector harvest. Samples were treated with DNAse for 2 hours at 37° C. and quantified by qPCR using primers and probe against EGFP transgene. Productivity of intact AAV2-EGFP was compared to helper-free transfection method (HF), wherein HEK293 cells were transfected with the AAV transfer genome plasmid (pAAV-EGFP), plasmid pRepCap2, and plasmid pHelper. Control samples were transfected with stuffer DNA (pUC19) in place of pRepCap2 to control for efficiency of DNAse treatment.

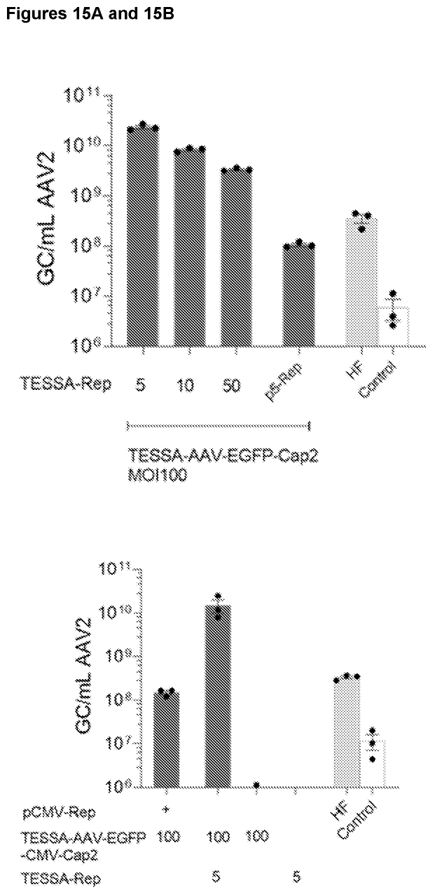

The result from FIG. 15A shows co-infection with two TERA vectors: TERA-Rep with TERA-AAV-EGFP-Cap2 produced significantly greater amounts of intact AAV2 vectors compared to suppling a Rep expression plasmid by transfection where the Rep is expressed from the native p5 AAV promoter or via the helper-free plasmids.

The results in FIG. 15B show the same experiment as that in FIG. 15A but where the p5 promoter driving Rep expression is replaced with a strong CMV promoter; this does not improve AAV productivity relative to the use of TERA-Rep.

### REFERENCES

Fisher, K. J. et al (1996) A novel adenovirus-adeno-associated virus hybrid vector that displays efficient rescue and delivery of the AAV genome. Hum gene ther. 7:2079-2087.
Liu, X. L et al (1999) Production of recombinant adeno-associated virus vectors using a packaging cell line and a hybrid recombinant adenovirus. Gene ther. 6: 293-299.
Sitaraman, V. et al (2011) Computationally designed adeno-associated virus (AAV) rep 78 is efficiently maintained within an adenovirus vector. Proc. Natl. Acad. Sci. 108:14294-14299.
Timpe, J. M. et al (2006) Effect of adeno-associated virus on adenovirus replication and gene expression during coinfection. J. Virol. 16:7807-7815.
Weger, S. et al (2016) A regulatory element near the 3′ end of adeno-associated virus rep gene inhibits adenovirus replication in cis by means of p40 promoter-associated short transcripts. J. Virol. 90: 3981-3993.
Zhang, H-G. et al (2001) Recombinant adenovirus expressing adeno-associated virus cap and rep proteins supports production of high-titre recombinant adeno-associated virus. Gene ther. 8: 704-712.
Zhang, X. and Li, C-Y. (2001) Generation of recombinant adeno-associated virus vectors by a complete adenovirus-mediated approach. Mol. Ther. 3: 787-693.

SEQUENCES
Rep nucleotide sequence (AAV serotype 2)
SEQ ID NO: 1
atgccggggttttacgagattgtgattaaggtccccagcgaccttgacgagcatctgcccggcatttctgacagctt
tgtgaactgggtggccgagaaggaatgggagttgccgccagattctgacatggatctgaatctgattgagcaggcac
ccctgaccgtggccgagaagctgcagcgcgactttctgacggaatggcgccgtgtgagtaaggccccggaggccctt
ttctttgtgcaatttgagaagggagagagctacttccacatgcacgtgctcgtggaaaccaccggggtgaaatccat
ggttttgggacgtttcctgagtcagattcgcgaaaaactgattcagagaatttaccgcgggatcgagccgactttgc
caaactggttcgcggtcacaaagaccagaaatggcgccggaggcgggaacaaggtggtggatgagtgctacatcccc
aattacttgctccccaaaacccagcctgagctccagtgggcgtggactaatatggaacagtatttaagcgcctgttt
gaatctcacggagcgtaaacggttggtggcgcagcatctgacgcacgtgtcgcagacgcaggagcagaacaaagaga
atcagaatcccaattctgatgcgccggtgatcagatcaaaaacttcagccaggtacatggagctggtcgggtggctc
gtggacaaggggattacctcggagaagcagtggatccaggaggaccaggcctcatacatctccttcaatgcggcctc
caactcgcggtcccaaatcaaggctgccttggacaatgcgggaaagattatgagcctgactaaaaccgcccccgact
acctggtgggccagcagcccgtggaggacatttccagcaatcggatttataaaattttggaactaaacgggtacgat
ccccaatatgcggcttccgtctttctgggatgggccacgaaaaagttcggcaagaggaacaccatctggctgtttgg
gcctgcaactaccgggaagaccaacatcgcggaggccatagcccacactgtgcccttctacgggtgcgtaaactgga
ccaatgagaactttcccttcaacgactgtgtcgacaagatggtgatctggtgggaggaggggaagatgaccgccaag
gtcgtggagtcggccaaagccattctcggaggaagcaaggtgcgcgtggaccagaaatgcaagtcctcggcccagat
agacccgactcccgtgatcgtcacctccaacaccaacatgtgcgccgtgattgacgggaactcaacgaccttcgaac
accagcagccgttgcaagaccggatgttcaaatttgaactcacccgccgtctggatcatgactttgggaaggtcacc
aagcaggaagtcaaagactttttccggtgggcaaaggatcacgtggttgaggtggagcatgaattctacgtcaaaaa
gggtggagccaagaaaagacccgcccccagtgacgcagatataagtgagcccaaacgggtgcgcgagtcagttgcgc
agccatcgacgtcagacgcggaagcttcgatcaactacgcagacaggtaccaaaacaaatgttctcgtcacgtgggc
atgaatctgatgctgtttccctgcagacaatgcgagagaatgaatcagaattcaaatatctgcttcactcacggaca
gaaagactgtttagagtgctttcccgtgtcagaatctcaacccgtttctgtcgtcaaaaaggcgtatcagaaactgt
gctacattcatcatatcatgggaaaggtgccagacgcttgcactgcctgcgatctggtcaatgtggatttggatgac
tgcatctttgaacaaTAG
Cap nucleotide sequence (AAV serotype 2)
SEQ ID NO: 2
Cagttgcgcagccatcgacgtcagacgcggaagcttcgatcaactacgcagacaggtaccaaaacaaatgttctcgt
cacgtgggcatgaatctgatgctgtttccctgcagacaatgcgagagaatgaatcagaattcaaatatctgcttcac
tcacggacagaaagactgtttagagtgctttcccgtgtcagaatctcaacccgtttctgtcgtcaaaaaggcgtatc
agaaactgtgctacattcatcatatcatgggaaaggtgccagacgcttgcactgcctgcgatctggtcaatgtggat
ttggatgactgcatctttgaacaataaatgatttaaatcaggt
atggctgccgatggttatcttccagattggctcgaggacactctctctgaaggaataagacagtggtggaagctcaa
acctggcccaccaccaccaaagcccgcagagcggcataaggacgacagcaggggtcttgtgcttcctgggtacaagt
acctoggacccttcaacggactcgacaagggagagccggtcaacgaggcagacgccgcggccctcgagcacgacaaa
gcctacgaccggcagctcgacagcggagacaacccgtacctcaagtacaaccacgccgacgcggagtttcaggagcg
cottaaagaagatacgtcttttgggggcaacctcggacgagcagtcttccaggcgaaaaagagggttcttgaacctc
tgggcctggttgaggaacctgttaagacggctccgggaaaaaagaggccggtagagcactctcctgtggagccagac
tcctcctcgggaaccggaaaggcgggccagcagcctgcaagaaaaagattgaattttggtcagactggagacgcaga
ctcagtacctgacccccagcctctcggacagccaccagcagccccctctggtctgggaactaatacgatggctacag
gcagtggcgcaccaatggcagacaataacgagggcgccgacggagtgggtaattcctcgggaaattggcattgcgat
tccacatggatgggcgacagagtcatcaccaccagcacccgaacctgggccctgcccacctacaacaaccacctcta
caaacaaatttccagccaatcaggagcctcgaacgacaatcactactttggctacagcaccccttgggggtattttg
acttcaacagattccactgccacttttcaccacgtgactggcaaagactcatcaacaacaactggggattccgaccc
aagagactcaacttcaagctctttaacattcaagtcaaagaggtcacgcagaatgacggtacgacgacgattgccaa
taaccttaccagcacggttcaggtgtttactgactcggagtaccagctcccgtacgtcctcggctcggcgcatcaag
gatgcctcccgccgttcccagcagacgtcttcatggtgccacagtatggatacctcaccctgaacaacgggagtcag
gcagtaggacgctcttcattttactgcctggagtactttccttctcagatgctgcgtaccggaaacaactttacctt
cagctacacttttgaggacgttcctttccacagcagctacgctcacagccagagtctggaccgtctcatgaatcctc
tcatcgaccagtacctgtattacttgagcagaacaaacactccaagtggaaccaccacgcagtcaaggcttcagttt
tctcaggccggagcgagtgacattcgggaccagtctaggaactggcttcctggaccctgttaccgccagcagcgagt
atcaaagacatctgcggataacaacaacagtgaatactcgtggactggagctaccaagtaccacctcaatggcagag
actctctggtgaatccgggcccggccatggcaagccacaaggacgatgaagaaaagttttttcctcagagcggggtt
ctcatctttgggaagcaaggctcagagaaaacaaatgtggacattgaaaaggtcatgattacagacgaagaggaaat
caggacaaccaatcccgtggctacggagcagtatggttctgtatctaccaacctccagagaggcaacagacaagcag
ctaccgcagatgtcaacacacaaggcgttcttccaggcatggtctggcaggacagagatgtgtaccttcaggggccc
atctgggcaaagattccacacacggacggacattttcacccctctcccctcatgggtggattcggacttaaacaccc
tcctccacagattctcatcaagaacaccccggtacctgcgaatccttcgaccaccttcagtgcggcaaagtttgctt
ccttcatcacacagtactccacgggacaggtcagcgtggagatcgagtgggagctgcagaaggaaaacagcaaacgc
tggaatcccgaaattcagtacacttccaactacaacaagtctgttaatgtggactttactgtggacactaatggcgt
gtattcagagcctcgccccattggcaccagatacctgactcgtaatctgtaA
Cap amino acid sequence (AAV serotype 2)
SEQ ID NO: 3
MAADGYLPDWLEDTLSEGIRQWWKLKPGPPPPKPAERHKDDSRGLVLPGYKYLGPFNGLDKGEPVNEADAAALEHDK
AYDRQLDSGDNPYLKYNHADAEFQERLKEDTSFGGNLGRAVFQAKKRVLEPLGLVEEPVKTAPGKKRPVEHSPVEPD
SSSGTGKAGQQPARKRLNFGQTGDADSVPDPQPLGQPPAAPSGLGTNTMATGSGAPMADNNEGADGVGNSSGNWHCD
STWMGDRVITTSTRTWALPTYNNHLYKQISSQSGASNDNHYFGYSTPWGYFDFNRFHCHFSPRDWQRLINNNWGFRP
KRLNFKLFNIQVKEVTQNDGTTTIANNLTSTVQVFTDSEYQLPYVLGSAHQGCLPPFPADVFMVPQYGYLTLNNGSQ
AVGRSSFYCLEYFPSQMLRTGNNFTFSYTFEDVPFHSSYAHSQSLDRLMNPLIDQYLYYLSRTNTPSGTTTQSRLQF
SQAGASDIRDQSRNWLPGPCYRQQRVSKTSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNRQAATADVNTQGVLPGMVWQDRDVYLQGP
IWAKIPHTDGHFHPSPLMGGFGLKHPPPQILIKNTPVPANPSTTFSAAKFASFITQYSTGQVSVEIEWELQKENSKR
WNPEIQYTSNYNKSVNVDFTVDTNGVYSEPRPIGTRYLTRNL*
SEQ ID NO: 4
gtcctgtattagaggtcacgtgagtgttttgcgacattttgcgacaccatgtggtcacgctgggtatttaagcccga
gtgagcacgcagggtctccattttgaagcgggaggtttgaacgcgcagccgcc
SEQ ID NO: 5
agatctttgtcgatcctaccatccactcgacacacccgccagcggccgctgccaagcttccgagctctcgaattc
SEQ ID NO: 6
gtcacaaagaccagaaatggcgccggaggcgggaacaaggtggtggatgagtgctacatccccaattacttgctccc
caaaacccagcctgagctccagtgggcgtggactaatatggaacagtatttaagcgcctgtttgaatctcacggagc
gtaaacggttggtggcgcagcatctgacgcacgtgtcgcagacgcaggagcagaacaaagagaatcagaatcccaat
tctgatgcgccggtgatcagatcaaaaacttcagccaggtac
SEQ ID NO: 7
gtcacaaagaccagaaatggcgccggaggcgggaacaaggtggtggatgagtgctacatccccaattacttgctccc
caaaacccagcctgagctccagtgggcgtggactaatatggaacagtacctcagcgcctgtttgaatctcacggagc
gtaaacggttggtggcgcagcatctgacgcacgtgtcgcagacgcaggagcagaacaaagagaatcagaatcccaat
tctgatgcgccggtgatcagatcaaaaacttcagccaggtac
SEQ ID NO: 8
Ggtcaccaagcaggaagtcaaagactttttccggtgggcaaaggatcacgtggttgaggtggagcatgaattctacg
tcaaaaagggtggagccaagaaaagacccgcccccagtgacgcagatataagtgagcccaaacgggtgcgcgagtca
gttgcgcagccatcgacgtcagacgcggaagcttcgatcaactacgcagacaggt
SEQ ID NO: 9
gtgacaaaacaagaggtgaaggacttctttcgttgggccaaagaccatgtggtcgaggtcgaacacgagttctatgt
gaagaaaggaggcgcgaagaagcgcccagcgccatcggacgctgacatctccgaaccgaagcgcgtgagagagagcg
tggcacaaccatcaacctcggatgccgaggcatccatcaattatgcggacaggt
SEQ ID NO: 10
Gtcaccaagcaggaagtcaaagactttttccggtgggcaaaggatcacgtggttgaggtggagcatgaattctacgt
caaaaagggtggagccaagaaaagacccgcccccagtgacgcagatataagtgagcccaaacgggtgcgcgagtcag
ttgcgcagccatcgacgtcagacgcggaagcttcgatcaactacgcagacaggt
SEQ ID NO: 11
gtgacaaaacaagaggtgaaggacttctttcgttgggccaaagaccatgtggtcgaggtcgaacacgagttctatgt
gaagaaaggaggcgcgaagaagcgcccagcgccatcggacgctgacatctccgaaccgaagcgcgtgagagagagcg
tggcacaaccatcaacctoggatgccgaggcatccatcaattatgcggacaggt
SEQ ID NO: 12
atgccggggttttacgagattgtgattaaggtccccagcgaccttgacgagcatctgcccggcatttctgacagctt
tgtgaactgggtggccgagaaggaatgggagttgccgccagattctgacatggatctgaatctgattgagcaggcac
ccctgaccgtggccgagaagctgcagcgcgactttctgacggaatggcgccgtgtgagtaaggccccggaggccctt
ttctttgtgcaatttgagaagggagagagctacttccacatgcacgtgctcgtggaaaccaccggggtgaaatccat
ggttttgggacgtttcctgagtcagattcgcgaaaaactgattcagagaatttaccgcgggatcgagccgactttgc
caaactggttcgcggtcacaaagaccagaaatggcgccggaggcgggaacaaggtggtggatgagtgctacatcccc
aattacttgctccccaaaacccagcctgagctccagtgggcgtggactaatatggaacagtacctcagcgcctgttt
gaatctcacggagcgtaaacggttggtggcgcagcatctgacgcacgtgtcgcagacgcaggagcagaacaaagaga
atcagaatcccaattctgatgcgccggtgatcagatcaaaaacttcagccaggtacatggagctggtcgggtggctc
gtggacaaggggaCtacctcggagaagcagtggatccaggaggaccaggcctcatacatctccttcaatgcggcctc
caactcgcggtcccaaatcaaggctgccttggacaatgcgggaaagattatgagcctgactaaaaccgcccccgact
acctggtgggccagcagcccgtggaggacatttccagcaatcggatttataaaattttggaactaaacgggtacgat
ccccaatatgcggcttccgtctttctgggatgggccacgaaaaagttcggcaagaggaacaccatctggctgtttgg
gcctgcaactaccgggaagaccaacatcgcggaggccatagcccacactgtgcccttctacgggtgcgtaaactgga
ccaatgagaactttcccttcaacgactgtgtcgacaagatggtgatctggtgggaggaggggaagatgaccgccaag
gtcgtggagtcggccaaagccattctcggaggaagcaaggtgcgcgtggaccagaaatgcaagtcctcggcccagat
agacccgactcccgtgatcgtcacctccaacaccaacatgtgcgccgtcatcgacggaaatagcaccactttcgagc
atcaacagcctctgcaggatcggatgtttaagttcgagctgacgaggcggctcgaccatgatttcgggaaagtgaca
aaacaagaggtgaaggacttctttcgttgggccaaagaccatgtggtcgaggtcgaacacgagttctatgtgaagaa
aggaggcgcgaagaagcgcccagcgccatcggacgctgacatctccgaaccgaagcgcgtgagagagagcgtggcac
aaccatcaacctcggatgccgaggcatccatcaattatgcggacaggtaccaaaacaaatgttctcgtcacgtgggc
atgaatctgatgctgtttccctgcagacaatgcgagagaatgaatcagaattcaaatatctgcttcactcacggaca
gaaagactgtttagagtgctttcccgtgtcagaatctcaacccgtttctgtcgtcaaaaaggcgtatcagaaactgt
gctacattcatcatatcatgggaaaggtgccagatgcttgcactgcctgcgatctggtcaatgtggatttggatgac
tgcatctttgaacaataaatgatttaattcaggtatggctgccgatggttatcttccagattggctcgaggacactc
tctctga
TetR binding site
SEQ ID NO: 13
tccctatcag tgatagaga
Modified MLP
SEQ ID NO: 14
cgccctcttc ggcatcaagg aaggtgattg gtttgtaggt gtaggccacg tgaccgggtg
ttcctgaagg ggggctataa aaggtcccta tcagtgatag agactca
Modified MLP
SEQ ID NO: 15
cgccctcttc ggcatcaagg aaggtgattg gtttgtaggt gtaggccacg tgactcccta
tcagtgatag agaactataa aaggtcccta tcagtgatag agactca
Rep52 nucleotide sequence
SEQ ID NO: 16
CATGGAGCTGGTCGGGTGGctcgtggacaaggggattacctcggagaagcagtggatccaggaggaccaggcctcat
acatctccttcaatgcggcctccaactcgcggtcccaaatcaaggctgccttggacaatgcgggaaagattatgagc
ctgactaaaaccgcccccgactacctggtgggccagcagcccgtggaggacatttccagcaatoggatttataaaat
tttggaactaaacgggtacgatccccaatatgcggcttccgtctttctgggatgggccacgaaaaagttcggcaaga
ggaacaccatctggctgtttgggcctgcaactaccgggaagaccaacatcgcggaggccatagcccacactgtgccc
ttctacgggtgcgtaaactggaccaatgagaactttcccttcaacgactgtgtcgacaagatggtgatctggtggga
ggaggggaagatgaccgccaaggtcgtggagtcggccaaagccattctcggaggaagcaaggtgcgcgtggaccaga
aatgcaagtcctcggcccagatagacccgactcccgtgatcgtcacctccaacaccaacatgtgcgccgtgattgac
gggaactcaacgaccttcgaacaccagcagccgttgcaagaccggatgttcaaatttgaactcacccgccgtctgga
tcatgactttgggaaggtcaccaagcaggaagtcaaagactttttccggtgggcaaaggatcacgtggttgaggtgg
agcatgaattctacgtcaaaaagggtggagccaagaaaagacccgcccccagtgacgcagatataagtgagcccaaa
cgggtgcgcgagtcagttgcgcagccatcgacgtcagacgcggaagcttcgatcaactacgcagacaggtaccaaaa
caaatgttctcgtcacgtgggcatgaatctgatgctgtttccctgcagacaatgcgagagaatgaatcagaattcaa
atatctgcttcactcacggacagaaagactgtttagagtgctttcccgtgtcagaatctcaacccgtttctgtcgtc
aaaaaggcgtatcagaaactgtgctacattcatcatatcatgggaaaggtgccagacgcttgcactgcctgcgatct
ggtcaatgtggatttggatgaCTGCATCTTTGAACAATAG
Rep40 nucleotide sequence
SEQ ID NO: 17
ATGGAGCTGGTCGGGTGGctcgtggacaaggggattacctcggagaagcagtggatccaggaggaccaggcctcata
catctccttcaatgcggcctccaactcgcggtcccaaatcaaggctgccttggacaatgcgggaaagattatgagcc
tgactaaaaccgcccccgactacctggtgggccagcagcccgtggaggacatttccagcaatoggatttataaaatt
ttggaactaaacgggtacgatccccaatatgcggcttccgtctttctgggatgggccacgaaaaagttcggcaagag
gaacaccatctggctgtttgggcctgcaactaccgggaagaccaacatcgcggaggccatagcccacactgtgccct
tctacgggtgcgtaaactggaccaatgagaactttcccttcaacgactgtgtcgacaagatggtgatctggtgggag
gaggggaagatgaccgccaaggtcgtggagtcggccaaagccattctcggaggaagcaaggtgcgcgtggaccagaa
atgcaagtcctcggcccagatagacccgactcccgtgatcgtcacctccaacaccaacatgtgcgccgtgattgacg
ggaactcaacgaccttcgaacaccagcagccgttgcaagaccggatgttcaaatttgaactcacccgccgtctggat
catgactttgggaaggtcaccaagcaggaagtcaaagactttttccggtgggcaaaggatcacgtggttgaggtgga
gcatgaattctacgtcaaaaagggtggagccaagaaaagacccgcccccagtgacgcagatataagtgagcccaaac
gggtgcgcgagtcagttgcgcagccatcgacgtcagacgcggaagcttcgatcaactacgcagacagattggctcga
ggacactctctcTAG
Rep52 amino acid sequence (AAV serotype 2)
SEQ ID NO: 18
MELVGWLVDKGITSEKQWIQEDQASYISFNAASNSRSQIKAALDNAGKIMSLTKTAPDYLVGQQPVEDISSNRIYKI
LELNGYDPQYAASVFLGWATKKFGKRNTIWLFGPATTGKTNIAEAIAHTVPFYGCVNWTNENFPFNDCVDKMVIWWE
EGKMTAKVVESAKAILGGSKVRVDQKCKSSAQIDPTPVIVTSNTNMCAVIDGNSTTFEHQQPLQDRMFKFELTRRLD
HDFGKVTKQEVKDFFRWAKDHVVEVEHEFYVKKGGAKKRPAPSDADISEPKRVRESVAQPSTSDAEASINYADRYQN
KCSRHVGMNLMLFPCRQCERMNQNSNICFTHGQKDCLECFPVSESQPVSVVKKAYQKLCYTHHIMGKVPDACTACDL
VNVDLDDCIFEQ*
Rep40 amino acid sequence (AAV serotype 2)
SEQ ID NO: 19
MELVGWLVDKGITSEKQWIQEDQASYISFNAASNSRSQIKAALDNAGKIMSLTKTAPDYLVGQQPVEDISSNRIYKI
LELNGYDPQYAASVFLGWATKKFGKRNTIWLFGPATTGKTNIAEAIAHTVPFYGCVNWTNENFPFNDCVDKMVIWWE
EGKMTAKVVESAKAILGGSKVRVDQKCKSSAQIDPTPVIVTSNTNMCAVIDGNSTTFEHQQPLQDRMFKFELTRRLD
HDFGKVTKQEVKDFFRWAKDHVVEVEHEFYVKKGGAKKRPAPSDADISEPKRVRESVAQPSTSDAEASINYADRLAR
GHSL*
Rep78 nucleotide sequence (AAV serotype 2)
SEQ ID NO: 20
atgccggggttttacgagattgtgattaaggtccccagcgaccttgacgggcatctgcccggcatttctgacagctt
tgtgaactgggtggccgagaaggaatgggagttgccgccagattctgacatggatctgaatctgattgagcaggcac
ccctgaccgtggccgagaagctgcagcgcgactttctgacggaatggcgccgtgtgagtaaggccccggaggccctt
ttctttgtgcaatttgagaagggagagagctacttccacatgcacgtgctcgtggaaaccaccggggtgaaatccat
ggttttgggacgtttcctgagtcagattcgcgaaaaactgattcagagaatttaccgcgggatcgagccgactttgc
caaactggttcgcggtcacaaagaccagaaatggcgccggaggcgggaacaaggtggtggatgagtgctacatcccc
aattacttgctccccaaaacccagcctgagctccagtgggcgtggactaatatggaacagtacctcagcgcctgttt
gaatctcacggagcgtaaacggttggtggcgcagcatctgacgcacgtgtcgcagacgcaggagcagaacaaagaga
atcagaatcccaattctgatgcgccggtgatcagatcaaaaacttcagccaggtacatggagctggtcgggtggctc
gtggacaaggggattacctcggagaagcagtggatccaggaggaccaggcctcatacatctccttcaatgcggcctc
caactcgcggtcccaaatcaaggctgccttggacaatgcgggaaagattatgagcctgactaaaaccgcccccgact
ccccaatatgcggcttccgtctttctgggatgggccacgaaaaagttcggcaagaggaacaccatctggctgtttgg
gcctgcaactaccgggaagaccaacatcgcggaggccatagcccacactgtgcccttctacgggtgcgtaaactgga
ccaatgagaactttcccttcaacgactgtgtcgacaagatggtgatctggtgggaggaggggaagatgaccgccaag
gtcgtggagtcggccaaagccattctcggaggaagcaaggtgcgcgtggaccagaaatgcaagtcctcggcccagat
agacccgactcccgtgatcgtcacctccaacaccaacatgtgcgccgtgattgacgggaactcaacgaccttcgaac
accagcagccgttgcaagaccggatgttcaaatttgaactcacccgccgtctggatcatgactttgggaaggtcacc
aagcaggaagtcaaagactttttccggtgggcaaaggatcacgtggttgaggtggagcatgaattctacgtcaaaaa
gggtggagccaagaaaagaGGGgcGcccagtgacgGagatataagtgagcccaaacgggtgGgGgagtcagttgcgc
agccatcgacgtcagacgcggaagcttcgatcaactacgcagacaggtaccaaaacaaatgttctcgtcacgtgggc
atgaatctgatgctgtttccctgcagacaatgcgagagaatgaatcagaattcaaatatctgcttcactcacggaca
gaaagactgtttagagtgctttcccgtgtcagaatctcaacccgtttctgtcgtcaaaaaggcgtatcagaaactgt
gctacattcatcatatcatgggaaaggtgccagacgcttgcactgcctgcgatctggtcaatgtggatttggatgac
tgcatctttgaacaaTAG
Rep68 nucleotide sequence (AAV serotype 2)
SEQ ID NO: 21
ATGCCGGGGTTTTACGAGattgtgattaaggtccccagcgaccttgacgagcatctgcccggcatttctgacagctt
tgtgaactgggtggccgagaaggaatgggagttgccgccagattctgacatggatctgaatctgattgagcaggcac
ccctgaccgtggccgagaagctgcagcgcgactttctgacggaatggcgccgtgtgagtaaggccccggaggccctt
ttctttgtgcaatttgagaagggagagagctacttccacatgcacgtgctcgtggaaaccaccggggtgaaatccat
ggttttgggacgtttcctgagtcagattcgcgaaaaactgattcagagaatttaccgcgggatcgagccgactttgc
caaactggttcgcggtcacaaagaccagaaatggcgccggaggcgggaacaaggtggtggatgagtgctacatcccc
aattacttgctccccaaaacccagcctgagctccagtgggcgtGGACTAATATGGAACAGTACCTCAGCGCCTGTTT
GAATCTCACGGagcgtaaacggttggtggcgcagcatctgacgcacgtgtcgcagacgcaggagcagaacaaagaga
atcagaatcccaattctgatgcgccggtgatcagatcaaaaacttcagccaggtacatggagctggtcgggtggctc
gtggacaaggggattacctcggagaagcagtggatccaggaggaccaggcctcatacatctccttcaatgcggcctc
caactcgcggtcccaaatcaaggctgccttggacaatgcgggaaagattatgagcctgactaaaaccgcccccgact
acctggtgggccagcagcccgtggaggacatttccagcaatcggatttataaaattttggaactaaacgggtacgat
ccccaatatgcggcttccgtctttctgggatgggccacgaaaaagttcggcaagaggaacaccatctggctgtttgg
gcctgcaactaccgggaagaccaacatcgcggaggccatagcccacactgtgcccttctacgggtgcgtaaactgga
ccaatgagaactttcccttcaacgactgtgtcgacaagatggtgatctggtgggaggaggggaagatgaccgccaag
gtcgtggagtcggccaaagccattctcggaggaagcaaggtgcgcgtggaccagaaatgcaagtcctcggcccagat
agacccgactcccgtgatcgtcacctccaacaccaacatgtgcgccgtgattgacgggaactcaacgaccttcgaac
accagcagccgttgcaagaccggatgttcaaatttgaactcacccgccgtctggatcatgactttgggaaggtcacc
aagcaggaagtcaaagactttttccggtgggcaaaggatcacgtggttgaggtggagcatgaattctacgtcaaaaa
gggtggagccaagaaaagacccgcccccagtgacgcagatataagtgagcccaaacgggtgcgcgagtcagttgcgc
agccatcgacgtcagacgcggaagcttcgatcaactacgcagacagTAG
Rep78 amino acid sequence (AAV serotype 2)
SEQ ID NO: 22
MPGFYEIVIKVPSDLDGHLPGISDSFVNWVAEKEWELPPDSDMDLNLIEQAPLTVAEKLQRDFLTEWRRVSKAPEAL
FFVQFEKGESYFHMHVLVETTGVKSMVLGRFLSQIREKLIQRIYRGIEPTLPNWFAVTKTRNGAGGGNKVVDECYIP
NYLLPKTQPELQWAWTNMEQYLSACLNLTERKRLVAQHLTHVSQTQEQNKENQNPNSDAPVIRSKTSARYMELVGWL
VDKGITSEKQWIQEDQASYISFNAASNSRSQIKAALDNAGKIMSLTKTAPDYLVGQQPVEDISSNRIYKILELNGYD
PQYAASVFLGWATKKFGKRNTIWLFGPATTGKTNIAEAIAHTVPFYGCVNWTNENFPFNDCVDKMVIWWEEGKMTAK
VVESAKAILGGSKVRVDQKCKSSAQIDPTPVIVTSNTNMCAVIDGNSTTFEHQQPLQDRMFKFELTRRLDHDFGKVT
KQEVKDFFRWAKDHVVEVEHEFYVKKGGAKKRPAPSDADISEPKRVRESVAQPSTSDAEASINYADRYQNKCSRHVG
MNLMLFPCRQCERMNQNSNICFTHGQKDCLECFPVSESQPVSVVKKAYQKLCYTHHIMGKVPDACTACDLVNVDLDD
CIFEQ*
Rep68 amino acid sequence (AAV serotype 2)
SEQ ID NO: 23
MPGFYEIVIKVPSDLDEHLPGISDSFVNWVAEKEWELPPDSDMDLNLIEQAPLTVAEKLQRDFLTEWRRVSKAPEAL
FFVQFEKGESYFHMHVLVETTGVKSMVLGRFLSQIREKLIQRIYRGIEPTLPNWFAVTKTRNGAGGGNKVVDECYIP
NYLLPKTQPELQWAWTNMEQYLSACLNLTERKRLVAQHLTHVSQTQEQNKENQNPNSDAPVIRSKTSARYMELVGWL
VDKGITSEKQWIQEDQASYISFNAASNSRSQIKAALDNAGKIMSLTKTAPDYLVGQQPVEDISSNRIYKILELNGYD
PQYAASVFLGWATKKFGKRNTIWLFGPATTGKTNIAEAIAHTVPFYGCVNWTNENFPFNDCVDKMVIWWEEGKMTAK
VVESAKAILGGSKVRVDQKCKSSAQIDPTPVIVTSNTNMCAVIDGNSTTFEHQQPLQDRMFKFELTRRLDHDFGKVT
KQEVKDFFRWAKDHVVEVEHEFYVKKGGAKKRPAPSDADISEPKRVRESVAQPSTSDAEASINYAD*
SEQUENCE LISTING FREE TEXT
<210> 1
<213> Rep nucleotide sequence (adeno-associated virus 2)
<210> 2
<213> Cap nucleotide sequence (adeno-associated virus 2)
<210> 3
<213> Cap amino acid sequence (adeno-associated virus 2)
<210> 4
<213> Wild-type adeno-associated virus 2 p5 promoter
<210> 5
<213> Sequence that forms part of the 5′-untranslated region (UTR) of the
Homo sapiens beta-globin gene
<210> 6
<213> Wild-type adeno-associated virus 2 p19 promoter
<210> 7
<223> Non-functional p19 sequence
<210> 8
<213> Wild-type adeno-associated virus 2 p40 promoter
<210> 9
<223> Non-functional p40 promoter sequence
<210> 10
<213> Wild-type adeno-associated virus 2 adenovirus inhibitor
<210> 11
<223> Nucleotide sequence without a functional p40 promoter sequence
<210> 12
<223> rep gene sequence with p19 and p40 promoters ablated, retaining
the Rep78 and Rep68 coding sequences
<210> 13
<223> TetR binding site
<210> 14
<223> Modified MLP
<210> 15
<223> Modified MLP
<210> 16
<223> Rep52 nucleotide sequence
<210> 17
<223> Rep40 nucleotide sequence
<210> 18
<213> Rep52 amino acid sequence (adeno-associated virus 2)
<210> 19
<213> Rep40 amino acid sequence (adeno-associated virus 2)
<210> 20
<213> Rep78 nucleotide sequence (adeno-associated virus 2)
<210> 21
<213> Rep68 nucleotide sequence (adeno-associated virus 2)
<210> 22
<213> Rep78 amino acid sequence (adeno-associated virus 2)
<210> 23
<213> Rep68 amino acid sequence (adeno-associated virus 2)

1. An adenoviral vector comprising a nucleic acid molecule, wherein the nucleotide sequence of the nucleic acid molecule encodes at least one AAV Rep polypeptide, wherein the Rep polypeptide-encoding sequence has the following features:
(i) it is not operably-associated with a functional AAV p5 promoter;
(ii) it does not comprise a functional AAV p19 promoter and/or it does not encode functional Rep52 and Rep40 polypeptides; and
(iii) it does not comprise a functional adenovirus inhibitor sequence.

2. An adenoviral vector as claimed in claim 1, wherein the nucleotide sequence encodes functional AAV Rep78 and Rep68 polypeptides.

3. An adenoviral vector as claimed in claim 1, wherein the nucleic acid molecule does not comprise a p5 promoter.

4. An adenoviral vector as claimed in claim 1, wherein the Rep polypeptide-encoding sequence has the features:
(i) it is not operably-associated with a functional AAV p5 promoter; and
(iii) it does not comprise a functional AAV p40 promoter and it does not comprise a functional adenovirus inhibitor sequence.

5. An adenoviral vector as claimed in claim 1, wherein the Rep 78 and/or Rep 68 polypeptides are only capable of being expressed at a low, baseline or minimal level.

6. An adenoviral vector as claimed in claim 1, wherein the Rep 52 and/or Rep 40 polypeptides are not capable of being expressed.

7. An adenoviral vector as claimed in claim 1, wherein the adenovirus inhibitor sequence is not capable of being transcribed.

8. An adenoviral vector claimed in claim 1, wherein the adenovirus inhibitor sequence is modified in such a way that the transcription of the adenovirus inhibitor sequence, in a host cell, would not inhibit the replication of a wild-type adenovirus in the host cell.

9. (canceled)

10. An adenoviral vector as claimed in claim 1, wherein the nucleic acid molecule is located into one of the adenoviral Early genes or inserted in a site from which one or more Early genes have been deleted.

11. An adenoviral vector as claimed in claim 10, wherein the nucleic acid molecule is located in the E1 region in the same direction of transcription as the E4, E2A and E2B expression cassettes.

12. An adenoviral vector as claimed in claim 1, wherein the nucleic acid molecule is not operably-associated with an upstream promoter.

13. An adenoviral vector as claimed in claim 1, wherein the adenoviral vector additionally comprises an AAV cap gene.

14. An adenoviral vector as claimed in claim 1, wherein the adenoviral vector additionally encodes a polypeptide which is capable of transcriptionally-activating a promoter which is not present in the adenoviral vector.

15. An adenoviral vector as claimed in claim 1, wherein the adenoviral vector comprises a repressible Major Late Promoter (MLP) or wherein the MLP comprises one or more repressor elements which are capable of regulating or controlling transcription of the adenoviral late genes, and wherein one or more of the repressor elements are located downstream of the MLP TATA box.

16. (canceled)

17. A kit comprising:
(A) a first adenoviral vector as claimed in claim 1, and
(B) second adenoviral vector comprising
(i) a nucleic acid molecule encoding an AAV Cap polypeptide, and/or
(ii) a nucleic acid molecule encoding a recombinant AAV genome.

18. A kit as claimed in claim 17, wherein the first adenoviral vector additionally encodes a polypeptide which is capable of transcriptionally-activating a promoter which is present in the second adenoviral vector or wherein the promoter in the second adenoviral vector is one which is operably-associated with an AAV cap gene in the second adenoviral vector.

19. (canceled)

20. (canceled)

21. A process for producing AAV particles, the process comprising the steps:
(a) infecting a mammalian host cell with a first adenoviral vector as claimed in claim 1;
(b) infecting the host cell with a second adenoviral vector, wherein the second adenoviral vector comprises a recombinant AAV genome comprising a transgene, wherein at least one of the first and second adenoviral vectors comprise an AAV cap gene;
(c) culturing the mammalian host cell in a culture medium under conditions such that AAV particles comprising the transgene are produced; and
(d) isolating or purifying AAV particles from the host cells or from the cell culture medium.

22. (canceled)

23. A process for producing AAV particles, the process comprising the steps:
(a) infecting a mammalian host cell with a first adenoviral vector as claimed in claim 1, wherein the mammalian host cell comprises a recombinant AAV genome stably integrated into the host cell genome, wherein the recombinant AAV genome comprises a transgene, and wherein:
(i) the adenoviral vector additionally comprises an AAV cap gene, or
(ii) an AAV cap gene is stably integrated into the mammalian host cell genome, or
(iii) the cell is infected with a second adenoviral vector comprising an AAV cap gene;
(b) culturing the mammalian host cell in a culture medium under conditions such that AAV particles comprising the transgene are produced; and
(c) isolating or purifying AAV particles from the cells or from the cell culture medium.

24. A process for producing recombinant AAV particles, the process comprising the steps:
(a) infecting a mammalian host cell with a first adenoviral vector as claimed in claim 1, wherein:
(i) the first adenoviral vector additionally comprises an AAV cap gene, or
(ii) the cell is infected with a second adenoviral vector comprising an AAV cap gene;
(b) infecting the mammalian host cell with a recombinant AAV comprising a transgene;
(c) culturing the mammalian host cell in a culture medium under conditions such that AAV particles comprising the transgene are produced; and
(d) isolating and/or purifying AAV particles from the cells or from the cell culture medium.

25. An adenoviral vector as claimed in claim 13, wherein the AAV cap gene is operably-associated with no promoter or a minimal promoter.


SEQUENCE LISTING (23 sequences)

SEQ ID NO: 1 - DNA, 1866 bp, Artificial Sequence
  Rep nucleotide sequence (adeno-associated virus 2)
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 2 - DNA, 2559 bp, Artificial Sequence
  Cap nucleotide sequence (adeno-associated virus 2)
cagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 3 - protein, 735 aa, Artificial Sequence [sequence omitted]

SEQ ID NO: 4 - DNA, 130 bp, Artificial Sequence
  Adeno-associated virus 2 p5 promoter
gtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 5 - DNA, 75 bp, Artificial Sequence
  Sequence that forms part of the 5′-UTR of the H.sapiens beta-globin gene
agatctttgtcgatcctaccatccactcgacacacccgccagcggccgctgccaagcttccgagctctcgaattc

SEQ ID NO: 6 - DNA, 273 bp, Artificial Sequence
  Adeno-associated virus 2 p19 promoter
gtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtac

SEQ ID NO: 7 - DNA, 273 bp, Artificial Sequence
  Non-functional p19 sequence
gtcacaaagaccagaaatggcgccggaggcgggaacaaggtggtggatgagtgctacatccccaattacttgctccccaaaacccagcctgagctccagtgggcgtggactaatatggaacagtacctcagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtac

SEQ ID NO: 8 - DNA, 209 bp, Artificial Sequence
  Adeno-associated virus 2 p40 promoter
ggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggt

SEQ ID NO: 9 - DNA, 208 bp, Artificial Sequence
  Non-functional p40 promoter sequence
gtgacaaaacaagaggtgaaggacttctttcgttgggccaaagaccatgtggtcgaggtcgaacacgagttctatgtgaagaaaggaggcgcgaagaagcgcccagcgccatcggacgctgacatctccgaaccgaagcgcgtgagagagagcgtggcacaaccatcaacctcggatgccgaggcatccatcaattatgcggacaggt

SEQ ID NO: 10 - DNA, 208 bp, Artificial Sequence
  Adeno-associated virus 2 adenovirus inhibitor
gtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggt

SEQ ID NO: 11 - DNA, 208 bp, Artificial Sequence; identical to SEQ ID NO: 9
  Nucleotide sequence without a functional p40 promoter sequence

SEQ ID NO: 12 - DNA, 1932 bp, Artificial Sequence
  Seq with ablated p19 and p40 promoters, and retained Rep78 and Rep68 coding seq
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtacctcagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtcatcgacggaaatagcaccactttcgagcatcaacagcctctgcaggatcggatgtttaagttcgagctgacgaggcggctcgaccatgatttcgggaaagtgacaaaacaagaggtgaaggacttctttcgttgggccaaagaccatgtggtcgaggtcgaacacgagttctatgtgaagaaaggaggcgcgaagaagcgcccagcgccatcggacgctgacatctccgaaccgaagcgcgtgagagagagcgtggcacaaccatcaacctcggatgccgaggcatccatcaattatgcggacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagatgcttgcactgcctgcgatctggtcaatgtggatttggatgactgcatctttgaacaataaatgatttaattcaggtatggctgccgatggttatcttccagattggctcgaggacactctctctga

SEQ ID NO: 13 - DNA, 19 bp, Artificial Sequence
  TetR binding site
tccctatcagtgatagaga

SEQ ID NO: 14 - DNA, 107 bp, Artificial Sequence
  Modified MLP
cgccctcttcggcatcaaggaaggtgattggtttgtaggtgtaggccacgtgaccgggtgttcctgaaggggggctataaaaggtccctatcagtgatagagactca

SEQ ID NO: 15 - DNA, 107 bp, Artificial Sequence
  Modified MLP
cgccctcttcggcatcaaggaaggtgattggtttgtaggtgtaggccacgtgactccctatcagtgatagagaactataaaaggtccctatcagtgatagagactca

SEQ ID NO: 16 - DNA, 1195 bp, Artificial Sequence
  Rep52 nucleotide sequence
catggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 17 - DNA, 939 bp, Artificial Sequence
  Rep40 nucleotide sequence
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagattggctcgaggacactctctctag

SEQ ID NO: 18 - protein, 397 aa, Artificial Sequence
  Rep52 amino acid sequence (adeno-associated virus 2)
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 19 - protein, 312 aa, Artificial Sequence
  Rep40 amino acid sequence (adeno-associated virus 2)
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 20 - DNA, 1866 bp, Artificial Sequence
  Rep78 nucleotide sequence (adeno-associated virus 2)
atgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtacctcagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 21 - DNA, 1589 bp, Artificial Sequence
  Rep68 nucleotide sequence (adeno-associated virus 2)
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtacctcagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagtag

SEQ ID NO: 22 - protein, 621 aa, Artificial Sequence
  Rep78 amino acid sequence (adeno-associated virus 2)
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 23 - protein, 528 aa, Artificial Sequence
  Rep68 amino acid sequence (adeno-associated virus 2)
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYAD